(19) United States
(12) Patent Application Publication
(10) Pub. No.: US 2001/0025045 A1
(43) Pub. Date: Sep. 27, 2001

(54) Substituted isoquinoline derivatives and their use as anticonvulsants

(75) Inventors: Peter David Edwards, Horsham (GB); John David Harling, Sawbridgeworth (GB); Mervyn Thompson, Harlow (GB)

Correspondence Address: GLAXOSMITHKLINE, Corporate Intellectual Property-UW2220, P.O. Box 1539, Kings of Prussia, PA, 19406-0939, US

(73) Assignee: SmithKline Beecham p.l.c.

(21) Appl. No.: 09/840,550

(22) Filed: Apr. 22, 2001

Related U.S. Application Data
(63) Continuation of application No. 09/530,027, filed on Jun. 7, 2000.

(30) Foreign Application Priority Data
Oct. 24, 1997 (GB) GB9722537.9
Dec. 17, 1997 (GB) GB9726663.9

(51) Int. Cl. A61K 31/655; A61K 31/55; A61K 31/47; C07D 223/16; C07D 217/22

(52) U.S. Cl. 514/307; 514/217.01; 514/151; 546/143; 540/594; 514/416; 548/470

(57) ABSTRACT

This invention relates to substituted isoquinoline derivatives and their use as anticonvulsants.

This invention relates to novel compounds, to processes for preparing them, and to their use as therapeutic agents.

U.S. Pat. No. 4,022,900 (Marion), FR-A-2004748 (Marion) and DE-A-2101691 (Marion) disclose benzamido-tetrahydroisoquinolines having anti-hypertensive and vasodilator properties, including the compound 5-(2,4,5-trimethoxy-benzamido)-2-methyl-1,2,3,4-tetrahydroisoquinoline, which can also be expressed as 2,4,5-trimethoxy-N-(2-methyl-1,2,3,4-tetrahydroisoquinolin-5-yl)benzamide.

WO 97/48683 discloses that benzamide compounds of formula (I) below possess anti-convulsant activity and are therefore believed to be useful in the treatment and/or prevention of anxiety, mania, depression, panic disorders and/or aggression, disorders associated with a subarachnoid haemorrhage or neural shock, the effects associated with withdrawal from substances of abuse such as cocaine, nicotine, alcohol and benzodiazepines, disorders treatable and/or preventable with anti-convulsive agents, such as epilepsy including post-traumatic epilepsy, Parkinson's disease, psychosis, migraine, cerebral ischaemia, Alzheimer's disease and other degenerative diseases such as Huntingdon's chorea, schizophrenia, obsessive compulsive disorders (OCD), neurological deficits associated with AIDS, sleep disorders (including circadian rhythm disorders, insomnia & narcolepsy), tics (e.g. Giles de la Tourette's syndrome), traumatic brain injury, tinnitus, neuralgia, especially trigeminal neuralgia, neuropathic pain, dental pain, cancer pain, inappropriate neuronal activity resulting in neurodysthesias in diseases such as diabetes, multiple sclerosis (MS) and motor neurone disease, ataxias, muscular rigidity (spasticity), temporomandibular joint dysfunction and amyotrophic lateral sclerosis (ALS).

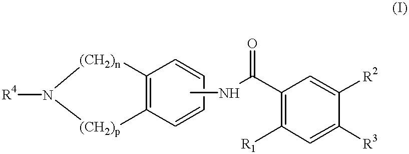

where n and p are independently integers from 1 to 4 and (n+p) is from 2 to 5;

R¹ is C₁₋₆alkylO—;

R² is hydrogen, halogen, CN, N₃, trifluoromethyldiazirinyl, CF₃, CF₃O—, CF₃S—, CF₃CO—, C₁₋₆alkyl, C₃₋₆cycloalkyl,C₃₋₆cycloalkyl—C₁₋₄alkyl—, C₁₋₆alkylO—, C₁₋₆alkylCO—, C₃₋₆cycloalkylCO—, C₃₋₆cycloalkyl—C₁₋₄alkylCO—, phenyl, phenoxy, benzyloxy, benzoyl, phenyl—C₁₋₄alkyl—, C₁₋₆alkylS—, C₁₋₆alkylSO₂—, (C₁₋₄alkyl)₂NSO₂— or (C₁₋₄alkyl)NHSO₂—;

R³ is hydrogen, halogen, NO₂, CN, N₃, trifluoromethyldiazirinyl, C₁₋₆ alkylO—, C₁₋₆ alkylS—, C₁₋₆ alkyl, C₃₋₆cycloalkyl, C₃₋₆cycloalkyl—C₁₋₄alkyl—, C₁₋₆alkenyl, C₁₋₆alkynyl, CF₃CO—, C₁₋₆alkylCO—, C₃₋₆cycloalkylCO—, C₃₋₆cycloalkyl—C₁₋₄alkylCO—, phenyl, phenoxy, benzyloxy, benzoyl, phenyl—C₁₋₄alkyl—, or —NR⁵R⁶ where R⁵ is hydrogen or C₁₋₄ alkyl, and

R⁶ is hydrogen, C₁₋₄alkyl, —CHO, —CO₂C₁₋₄alkyl or —COC₁₋₄alkyl;

R⁴ is hydrogen, C₁₋₆ alkyl, C₁₋₆ alkenyl, or C₁₋₆ alkynyl.

This invention is based on the finding that additional carboxamides possess anti-convulsant activity and are therefore believed to be useful in the treatments listed above.

According to a first aspect of the present invention, there is provided a compound of formula (Ia) or pharmaceutically acceptable salt thereof:

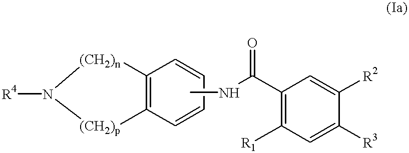

where n and p are independently integers from 1 to 4 and (n+p) is from 2 to 5;

R¹ is hydrogen, C₃₋₆cycloalkylO— or C₃₋₆cycloalkyl C₁₋₄alkylO—;

R² is hydrogen, halogen, CN, N₃, trifluoromethyldiazirinyl, C₁₋₆perfluoroalkyl, CF₃O—, CF₃S—, CF₃CO—, C₁₋₆alkyl, C₃₋₆cycloalkyl, CF₃SO₂—, C₃₋₆cycloalkyl—C₁₋₄alkyl—, C₁₋₆alkylO—, C₁₋₆alkylCO—, C₃₋₆—, ₆cycloalkylCO—, C₃₋₆cycloalkyl—C₁₋₄alkylCO—, phenyl, phenoxy, benzyloxy, benzoyl, phenyl—C₁₋₄alkyl—, C₁₋₆alkylS—, C₁₋₆alkylSO₂—, (C₁₋₄alkyl)₂NSO₂, (C₁₋₄alkyl)NHSO₂, (C₁₋₄alkyl)₂NCO—, (C₁₋₄alkyl)NHCO—;or CONH₂

R³ is hydrogen, halogen, NO₂, CN, N₃, trifluoromethyldiazirinyl, C₁₋₆ alkylO—, C₁₋₆ alkylS—, C₁₋₆ alkyl, C₁₋₆ hydroxyalkyl, C₃₋₆cycloalkyl, C₃₋₆cycloalkyl—C₁₋₄alkyl—, C₁₋₆alkenyl, C₁₋₆alkynyl, CF₃CO—, C₁₋₆alkylCO—, C₃₋₆cycloalkylCO—, C₃₋₆cycloalkyl—C₁₋₄alkylCO—, phenyl, phenoxy, benzyloxy, benzoyl, phenyl—C₁₋₄alkyl—, or —NR⁵R⁶ where R⁵ is hydrogen or C₁₋₄ alkyl, and R⁶ is hydrogen, C₁₋₄alkyl, —CHO, —CO₂C₁₋₄alkyl or —COC₁₋₄alkyl; or

R² and R³ together form a carbocyclic ring that is unsaturated or saturated and unsubstituted or substituted by carbonyl or hydroxyl;

R⁴ is hydrogen, C₁₋₆ alkyl,optionally substituted by hydroxy or C₁₋₄alkoxy; C₁₋₆ alkenyl, or C₁₋₆ alkynyl, formyl, C₁₋₆alkylCO, C₁₋₆alkylSO₂, or CF₃CO—.

According to a second aspect of the present invention, there is provided a compound of formula (Ib) or pharmaceutically acceptable salt thereof:

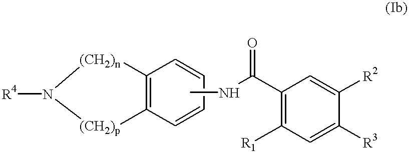

where n and p are independently integers from 1 to 4 and (n+p) is from 2 to 5;

R¹ is hydrogen, C₁₋₆alkylO—, C₃₋₆cycloalkylO— or C₃₋₆cycloalkyl C₁₋₄alkylO—;

R² is C₂₋₆perfluoroalkyl, (C₁₋₄alkyl)₂NCO—,(C₁₋₄alkyl)NHCO—; CF₃SO₂—,or CONH₂

R³ is hydrogen, halogen, NO₂, CN, N₃, trifluoromethyldiazirinyl, C₁₋₆ alkylO—, C₁₋₆ alkylS—, C₁₋₆ alkyl, C₁₋₆ hydroxyalkyl, C₃₋₆cycloalkyl, C₃₋₆cycloalkyl—C₁₋₄alkyl—, C₁₋₆alkenyl, C₁₋₆alkynyl, CF₃CO—, C₁₋₆alkylCO—, C₃₋₆cycloalkylCO—, C₃₋₆cycloalkyl—C₁₋₄alkylCO—, phenyl, phenoxy, benzyloxy, benzoyl, phenyl—C₁₋₄alkyl—, or —NR⁵R⁶ where R⁵ is hydrogen or C₁₋₄ alkyl, and R⁶ is hydrogen, C₁₋₄alkyl, —CHO, —CO₂C_{1 4}alkyl or —COC₁₋₄alkyl;

R⁴ is hydrogen, C₁₋₆ alkyl,optionally substituted by hydroxy or C₁₋₄alkoxy; C₁₋₆ alkenyl, or C₁₋₆ alkynyl, formyl, C₁₋₆alkylCO , C₁₋₆alkylSO₂, or CF₃CO—.

According to a third aspect of the present invention, there is provided a compound of formula (Ic) or pharmaceutically acceptable salt thereof:

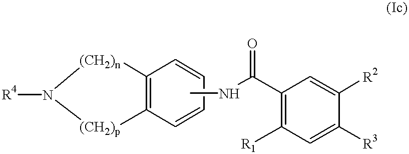

where n and p are independently integers from 1 to 4 and (n+p) is from 2 to 5;

R¹ is hydrogen, C₁₋₆alkylO—, C₃₋₆cycloalkylO— or C₃₋₆cycloalkyl C₁₋₄alkylO—;

R² and R³ together form a carbocyclic ring that is unsaturated or saturated and unsubstituted or substituted by carbonyl or hydroxyl;

R⁴ is hydrogen, C₁₋₆ alkyl,optionally substituted by hydroxy or C₁₋₄alkoxy; C₁₋₆ alkenyl, or C₁₋₆ alkynyl, formyl, C₁₋₆alkylCO, C₁₋₆alkylSO₂, or CF₃CO—.

According to a fourth aspect of the present invention, there is provided a compound of formula (Id) or pharmaceutically acceptable salt thereof:

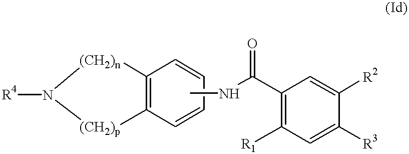

where n and p arc independently integers from 1 to 4 and (n+p) is from 2 to 5;

R¹ is hydrogen, C₁₋₆alkylO—, C₃₋₆cycloalkylO— or C₃₋₆cycloalkyl C₁₋₄alkylO—;

R² is hydrogen, halogen, CN, N₃, trifluoromethyldiazirinyl, C₁₋₆perfluoroalkyl, CF₃SO₂—,CF₃O—, CF₃S—, CF₃CO—, C₁₋₆alkyl, C₃₋₆cycloalkyl, C₃₋₆cycloalkyl—C₁₋₄alkyl—, C₁₋₆alkylO—, C₁₋₆alkylCO—, C₃₋₆cycloalkylCO—, C₃₋₆cycloalkyl—C₁₋₄alkylCO—, phenyl, phenoxy, benzyloxy, benzoyl, phenyl—C₁₋₄alkyl—, C₁₋₆alkylS—, C₁₋₆alkylSO₂—, (C₁₋₄alkyl)₂NSO₂, (C₁₋₄alkyl)NHSO₂, (C₁₋₄alkyl)₂NCO—, (C₁₋₄alkyl)NHCO—;or CONH₂

R³ is hydrogen, halogen, NO₂, CN, N₃, trifluoromethyldiazirinyl, C₁₋₆ alkylO—, C₁₋₆ alkylS—, C₁₋₆ alkyl, C₁₋₆ hydroxyalkyl, C₃₋₆cycloalkyl, C₃₋₆cycloalkyl—C₁₋₄alkyl—, C₁₋₆alkenyl, C₁₋₆alkynyl, CF₃CO—, C₁₋₆alkylCO—, C₃₋₆cycloalkylCO—, C₃₋₆cycloalkyl—C₁₋₄alkylCO—, phenyl, phenoxy, benzyloxy, benzoyl, phenyl—C₁₋₄alkyl—, or —NR⁵R⁶ where R⁵ is hydrogen or C₁₋₄ alkyl, and

R⁶ is hydrogen, C₁₋₄alkyl, —CHO, —CO₂C₁₋₄alkyl or —COC₁₋₄alkyl; or

R² and R³ together form a carbocyclic ring that is unsaturated or saturated and unsubstituted or substituted by carbonyl or hydroxyl;

R⁴ is C₁₋₆ alkyl substituted by hydroxy or C₁₋₄alkoxy; formyl, C₁₋₆alkylCO, C₁₋₆alkylSO₂,or CF₃CO—.

The compounds of this invention may be N-tetrahydroisoquinolinyl carboxamides, typically N-(tetrahydroisoquinolin-5-yl)carboxamides, N-(tetrahydroisoquinolin-6-yl)carboxamides, N-(tetrahydroisoquinolin-7-yl)carboxamides or N-(tetrahydroisoquinolin-8-yl)carboxamides, especially N-(tetrahydroisoquinolin-7-yl)carboxamides, and most suitably N-(tetrahydroisoquinolin-5-yl)carboxamides; or N-dihydroisoindolyl carboxamides, typically N-(dihydroisoindol-4-yl)carboxamides; or N-tetrahydrobenzazepinyl carboxamides, typically N-(tetrahydro-3-benzazepin-8-yl)carboxamides.

The carboxamide moiety may be a benzamide. When R² and R³ form a carbocyclic ring, this is typically a 5-7 membered ring, and the carboxamide moiety may be a naphthalene carboxamide or an indane or indanone carboxamide.

In the formula (Ia), (Ib), (Ic) or (Id), R¹ alkoxy groups are typically based on straight chain alkyl groups, but in general alkyl groups may be straight chain or branched. Aromatic rings, such as the aromatic ring in the bicyclic heterocyclic moiety in formula (I) and phenyl groups, including phenyl groups that are part of other moieties, in R² and R³ may optionally be substituted with one or more independently selected halogen or C₁₋₆ alkyl, C₁₋₆ alkoxy or C₁₋₆ alkylcarbonyl.

Suitable C₃₋₆ cycloalkyl groups include cyclopropyl, cyclobutyl, cyclopentyl, and cyclohexyl.

Suitable halo substituents include fluoro, chloro, iodo and bromo.

A suitable group of compounds of formula (Ia) have

R¹ as cyclopropylmethoxy;

R² as hydrogen, methoxy, bromo, chloro, iodo, acetyl, pivaloyl, iso-butyroyl, benzoyl, trifluoromethyl, trifluoroacetyl, methanesulfonyl, n-propylsulfonyl, isopropylsulfonyl, dimethylsulfamoyl or perfluoroethyl;

R³ as hydrogen, methyl, ethyl, n-butyl, iso-propyl, t-butyl, phenyl; methoxy, ethoxy, iso-propoxy, n-butoxy, phenoxy, benzyloxy, amino, acetylamino, nitro, benzoyl, iodobenzoyl, chloro or azido; or

R² and R³ form a benzene, cyclopentane or cyclopentanone ring;

R⁴ as hydrogen, methyl, ethyl, propyl, acetyl, trifluoromethylcarbonyl or methanesulfonyl.

A suitable group of compounds of formula (Ib) have

R¹ as methoxy, ethoxy, n-propoxy or cyclopropylmethoxy;

R² as methanesulfonyl or perfluoroethyl;

R³ as hydrogen, methyl, ethyl, n-butyl, iso-propyl, t-butyl, phenyl; methoxy, ethoxy, iso-propoxy, n-butoxy, phenoxy, benzyloxy, amino, acetylamino, nitro, benzoyl, iodobenzoyl, chloro or azido;

R⁴ as hydrogen, methyl, ethyl, propyl, acetyl, trifluoromethylcarbonyl or methanesulfonyl.

A suitable group of compounds of formula (Ic) have

R¹ as methoxy, ethoxy, n-propoxy or cyclopropylmethoxy;

R² and R³ form a benzene, cyclopentane or cyclopentanone ring;

R⁴ as hydrogen, methyl, ethyl, propyl, acetyl, trifluoromethylcarbonyl or methanesulfonyl.

A suitable group of compounds of formula (Id) have

R¹ as methoxy, ethoxy, n-propoxy or cyclopropylmethoxy;

R² as hydrogen, methoxy, bromo, chloro, iodo, acetyl, pivaloyl, iso-butyroyl, benzoyl, trifluoromethyl, trifluoroacetyl, methanesulfonyl, n-propylsulfonyl. isopropylsulfonyl, dimethylsulfamoyl or perfluoroethyl;

R³ as hydrogen, methyl, ethyl, n-butyl, iso-propyl, t-butyl, phenyl; methoxy, ethoxy, iso-propoxy, n-butoxy, phenoxy, benzyloxy, amino, acetylamino, nitro, benzoyl, iodobenzoyl, chloro or azido; or

R² and R³ form a benzene, cyclopentane or cyclopentanone ring;

R⁴ as acetyl, trifluoroacetyl or methanesulfonyl.

Examples of compounds of formulae (Ia) to (Id) are set out hereinbelow in the Examples.

Additionally the present invention provides the following novel compounds of formula (I):

N-(2-methyl-1,2,3,4-tetrahydroisoquinolin-5-yl)-5-iso-butyroyl-2-methoxy4-iso-propoxy-benzamide

N-(2-methyl-1,2,3,4-tetrahydroisoquinolin-5-yl)-5-acetyl-2-methoxy4-iso-propyl-benzamide

N-(2-methyl-1,2,3,4-tetrahydroisoquinolin-5-yl)-5-cyano-2-methoxy4-iso-propyl-benzamide

N-(2-methyl-1,2,3,4-tetrahydroisoquinolin-5-yl)-2-ethoxy-4-iso-propyl-5-trifluoromethyl-benzamide

N-(2-methyl-1,2,3,4-tetrahydroisoquinolin-5-yl)-2-n-propoxy-4-iso-propyl-5-trifluoromethyl-benzamide

N-(2-methyl-1,2,3,4-tetrahydroisoquinolin-5-yl)-2-iso-propoxy-4-iso-propyl-5-trifluoromethyl-benzamide

N-(2-methyl-1,2,3,4-tetrahydroisoquinolin-5-yl)-2-n-butoxy-4-iso-propyl-5-trifluoromethyl-benzamide

N-(2-methyl-1,2,3,4-tetrahydroisoquinolin-5-yl)-5-cyano-2-ethoxy-4-iso-propyl-benzamide

N-(2-methyl-1,2,3,4-tetrahydroisoquinolin-5-yl)-5-bromo4-ethoxy-2-methoxy-benzamide

N-(2-methyl-1,2,3,4-tetrahydroisoquinolin-5-yl)-4-ethoxy-2-methoxy-5-trifluoromethyl-benzamide

N-(2-methyl-1,2,3,4-tetrahydroisoquinolin-7-yl)-2,4-dimethoxy-5-trifluoromethyl-benzamide

N-(2-methyl-1,2,3,4-tetrahydroisoquinolin-5-yl)-4-n-butoxy-2-methoxy-5-cyanobenzamide

N-(2-methyl-1,2,3,4-tetrahydroisoquinolin-5-yl)-4-ethoxy-2-methoxy-5-cyanobenzamide

N-(2-methyl-1,2,3,4-tetrahydroisoquinolin-5-yl)-2,4-dimethoxy-5-cyanobenzamide

N-(2-methyl-1,2,3,4-tetrahydroisoquinolin-5-yl)-2-methoxy-4-iso-propoxy-5-cyanobenzamide

N-(2-methyl-1,2,3,4-tetrahydroisoquinolin-5-yl)-2,4-diethoxy-5-bromobenzamide.

When synthesised, these compounds are often in salt form, typically the hydrochloride or trifluoroacetate, and such salts also form part of this invention. Such salts may be used in preparing pharmaceutically acceptable salts. The compounds and their salts may be obtained as solvates, such as hydrates, and these also form part of this invention.

The above-listed compounds and pharmaceutically acceptable salts thereof, especially the hydrochloride, and pharmaceutically acceptable solvates, especially hydrates, form a preferred aspect of the present invention.

The administration of such compounds to a mammal may be by way of oral, parenteral, sub-lingual, nasal, rectal or transdermal administration.

An amount effective to treat the disorders hereinbefore described depends on the usual factors such as the nature and severity of the disorders being treated and the weight of the mammal. However, a unit dose will normally contain 1 to 1000 mg, suitably 1 to 500 mg, for example an amount in the range of from 2 to 400 mg such as 2, 5, 10, 20, 30, 40, 50, 100, 200, 300 and 400 mg of the active compound. Unit doses will normally be administered once or more than once per day, for example 1, 2, 3, 4, 5 or 6 times a day, more usually 1 to 4 times a day, such that the total daily dose is normally in the range, for a 70 kg adult of 1 to 1000 mg, for example 1 to 500 mg, that is in the range of approximately 0.01 to 15 mg/kg/day, more usually 0.1 to 6 mg/kg/day, for example 1 to 6 mg/kg/day.

It is greatly preferred that the compound of formula (I) is administered in the form of a unit-dose composition, such as a unit dose oral (including sub-lingual), nasal, rectal, topical or parenteral (especially intravenous) composition.

Such compositions are prepared by admixture and are suitably adapted for oral or parenteral administration, and as such may be in the form of tablets, capsules, oral liquid preparations, powders, granules, lozenges, reconstitutable powders, injectable and infusable solutions or suspensions or suppositories. Orally administrable compositions are preferred, in particular shaped oral compositions, since they are more convenient for general use.

Tablets and capsules for oral administration are usually presented in a unit dose, and contain conventional excipients such as binding agents, fillers, diluents, tabletting agents, lubricants, disintegrants, colourants, flavourings, and wetting agents. The tablets may be coated according to well known methods in the art.

Suitable fillers for use include cellulose, mannitol, lactose and other similar agents. Suitable disintegrants include starch, polyvinylpyrrolidone and starch derivatives such as sodium starch glycollate. Suitable lubricants include, for example, magnesium stearate. Suitable pharmaceutically acceptable wetting agents include sodium lauryl sulphate.

These solid oral compositions may be prepared by conventional methods of blending, filling, tabletting or the like. Repeated blending operations may be used to distribute the active agent throughout those compositions employing large quantities of fillers. Such operations are, of course, conventional in the art.

Oral liquid preparations may be in the form of, for example, aqueous or oily suspensions, solutions, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethyl cellulose, aluminium stearate gel or hydrogenated edible fats, emulsifying agents, for example lecithin, sorbitan monooleate, or acacia; non-aqueous vehicles (which may include edible oils), for example, almond oil, fractionated coconut oil, oily esters such as esters of glycerine, propylene glycol, or ethyl alcohol; preservatives, for example methyl or propyl p-hydroxybenzoate or sorbic acid, and if desired conventional flavouring or colouring agents.

Oral formulations also include conventional sustained release formulations, such as tablets or granules having an enteric coating.

For parenteral administration, fluid unit dose forms are prepared containing the compound and a sterile vehicle. The compound, depending on the vehicle and the concentration, can be either suspended or dissolved. Parenteral solutions are normally prepared by dissolving the compound in a vehicle and filter sterilising before filling into a suitable vial or ampoule and sealing. Advantageously, adjuvants such as a local anaesthetic, preservatives and buffering agents are also dissolved in the vehicle. To enhance the stability, the composition can be frozen after filling into the vial and the water removed under vacuum.

Parenteral suspensions are prepared in substantially the same manner except that the compound is suspended in the vehicle instead of being dissolved and sterilised by exposure to ethylene oxide before suspending in the sterile vehicle. Advantageously, a surfactant or wetting agent is included in the composition to facilitate uniform distribution of the compound of the invention.

As is common practice, the compositions will usually be accompanied by written or printed directions for use in the medical treatment concerned.

Accordingly, in a further aspect, the present invention provides a pharmaceutical composition for use in the treatment and/or prophylaxis of anxiety, mania, depression, panic disorders and/or aggression, disorders associated with a subarachnoid haemorrhage or neural shock, the effects associated with withdrawal from substances of abuse such as cocaine, nicotine, alcohol and benzodiazepines, disorders treatable and/or preventable with anti-convulsive agents, such as epilepsy including post-traumatic epilepsy, Parkinson's disease, psychosis, migraine, cerebral ischaemia, Alzheimer's disease and other degenerative diseases such as Huntingdon's chorea, schizophrenia, obsessive compulsive so disorders (OCD), neurological deficits associated with AIDS, sleep disorders (including circadian rhythm disorders, insomnia & narcolepsy), tics (e.g. Giles de la Tourette's syndrome), traumatic brain injury, tinnitus, neuralgia, especially trigeminal neuralgia, neuropathic pain, dental pain, cancer pain, inappropriate neuronal activity resulting in neurodysthesias in diseases such as diabetes, multiple sclerosis (MS) and motor neurone disease, ataxias, muscular rigidity (spasticity), temporomandibular joint dysfunction and/or amyotrophic lateral sclerosis (ALS), which comprises a compound of this invention, or a pharmaceutically acceptable salt or solvate thereof, and a pharmaceutically acceptable carrier.

The present invention also provides a method of treatment and/or prophylaxis of anxiety, mania, depression, panic disorders and/or aggression, disorders associated with a subarachnoid haemorrhage or neural shock, the effects associated with withdrawal from substances of abuse such as cocaine, nicotine, alcohol and benzodiazepines, disorders treatable and/or preventable with anti-convulsive agents, such as epilepsy including post-traumatic epilepsy, Parkinson's disease, psychosis, migraine, cerebral ischaemia, Alzheimer's disease and other degenerative diseases such as Huntingdon's chorea, schizophrenia, obsessive compulsive disorders (OCD), neurological deficits associated with AIDS, sleep disorders (including circadian rhythm disorders, insomnia & narcolepsy), tics (e.g. Giles de la Tourette's syndrome), traumatic brain injury, tinnitus, neuralgia, especially trigeminal neuralgia, neuropathic pain, dental pain, cancer pain, inappropriate neuronal activity resulting in neurodysthesias in diseases such as diabetes, multiple sclerosis (MS) and motor neurone disease, ataxias, muscular rigidity (spasticity), temporomandibular joint dysfunction and/or amyotrophic lateral sclerosis (ALS), comprising administering to the sufferer in need thereof an effective or prophylactic amount of a compound of this invention, or a pharmaceutically acceptable salt or solvate thereof.

In a further aspect the invention provides the use of a compound of this invention, or a pharmaceutically acceptable salt or solvate thereof, for the manufacture of a medicament for the treatment and/or prophylaxis of anxiety, mania, depression, panic disorders and/or aggression, disorders associated with a subarachnoid haemorrhage or neural shock, the effects associated with withdrawal from substances of abuse such as cocaine, nicotine, alcohol and benzodiazepines, disorders treatable and/or preventable with anti-convulsive agents, such as epilepsy including post-traumatic epilepsy, Parkinson's disease, psychosis, migraine, cerebral ischaemia, Alzheimer's disease and other degenerative diseases such as Huntingdon's chorea, schizophrenia, obsessive compulsive disorders (OCD), neurological deficits associated with AIDS, sleep disorders (including circadian rhythm disorders, insomnia & narcolepsy), tics (e.g. Giles de la Tourette's syndrome), traumatic brain injury, tinnitus, neuralgia, especially trigeminal neuralgia, neuropathic pain, dental pain, cancer pain, inappropriate neuronal activity resulting in neurodysthesias in diseases such as diabetes, multiple sclerosis (MS) and motor neurone disease, ataxias, muscular rigidity (spasticity), temporomandibular joint dysfunction and/or amyotrophic lateral sclerosis (ALS).

In a further aspect the invention provides the use of a compound of this invention, or a pharmaceutically acceptable salt or solvate, thereof as a therapeutic agent, in particular for the treatment and/or prophylaxis of anxiety, mania, depression, panic disorders and/or aggression, disorders associated with a subarachnoid haemorrhage or neural shock, the effects associated with withdrawal from substances of abuse such as cocaine, nicotine, alcohol and benzodiazepines, disorders treatable and/or preventable with anti-convulsive agents, such as epilepsy including post-traumatic epilepsy, Parkinson's disease, psychosis, migraine, cerebral ischaemia, Alzheimer's disease and other degenerative diseases such as Huntingdon's chorea, schizophrenia, obsessive compulsive disorders (OCD), neurological deficits associated with AIDS, sleep disorders (including circadian rhythm disorders, insomnia & narcolepsy), tics (e.g. Giles de la Tourette's syndrome), traumatic brain injury, tinnitus, neuralgia, especially trigeminal neuralgia, neuropathic pain, dental pain, cancer pain, inappropriate neuronal activity resulting in neurodysthesias in diseases such as diabetes, multiple sclerosis (MS) and motor neurone disease, ataxias, muscular rigidity (spasticity), temporomandibular joint dysfunction and/or amyotrophic lateral sclerosis (ALS).

Another aspect of the invention provides a process for the preparation of compounds of formula (Ia), (Ib), (Ic) or (Id), which comprises reacting a compound of formula (II)

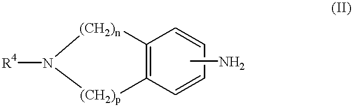

where n and p are as defined for formula (Ia), (Ib), (Ic) or (Id) and R^{4A} is R⁴ as defined for formula (Ia), (Ib), (Ic) or (Id) or a group convertible to R⁴ with a compound of formula (D)

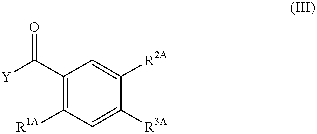

where Y is Cl or OH, and R^{1A}, R^{2A}, and R^{3A} are respectively R¹, R², and R³ as defined for formula (Ia), (Ib), (Ic) or (Id) or groups convertible to R¹, R², and R³, and where required converting a R^{1A}, R^{2A}, R^{3A} or R^{4A} group to a R¹, R², R³ or R⁴ group, converting one R¹, R², R³ or R⁴ group to another R¹, R², R³ or R⁴ group, converting a hydrochloride salt product to the free base or another pharmaceutically acceptable salt or converting a free base product to a pharmaceutically acceptable salt.

Reaction of a compound of formula (III) which is a benzoyl chloride derivative (Y═Cl) will lead directly to the hydrochloride salt. Suitable solvents include ethyl acetate and tetrahydrofuran. When the compound of formula (III) is a benzoic acid derivative (Y═OH), conventional conditions for condensation of aromatic acids with amines may be used, for example reacting the components in a mixture of (dimethylaminopropyl)-ethyl-carbodiimide and hydroxybenzotriazole in a suitable solvent such as dimethyl formamide.

Conversions of an R^{1A}, R^{2A}, R^{3A} or R^{4A} group to a R¹, R², R³ or R⁴ group typically arise when a protecting group is needed during the above coupling reaction or during the preparation of the reactants by the procedures described below. Interconversion of one R¹, R², R³ or R⁴ group to another typically arises when one compound of formula (Ia), (Ib), (Ic) or(Id) is used as the immediate precursor of another compound of formula (Ia), (Ib), (Ic) or(Id) or when it is easier to introduce a more complex or reactive substituent at the end of a synthetic sequence.

Compounds of formula (II) may be prepared from a compound of formula (IV)

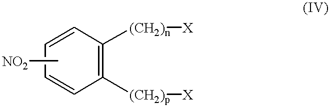

where X is a leaving group, such as halogen, especially bromo, or methanesulfonyl, which is reacted with R^{4A}NH₂, where R^{4A} is R⁴ as defined above or an N-protecting group, to obtain compounds of formula (V)

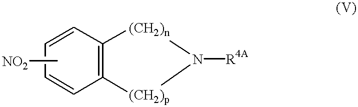

and then reduced, for example using hydrogen/palladium, to obtain compounds of formula (II).

Alternatively, a compound of formula (VI)

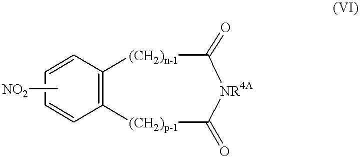

may be reduced directly, for example with lithium aluminium hydride, typically in tetrahydrofuran, to obtain a compound of formula (II) or a compound of formula (II) may be obtained in a two step procedure where a hydrogenation, typically with hydrogen/palladium, is followed by reduction, again suitably with lithium aluminium hydride.

When R^{4A} in formula (V) or (VI) is alkenyl or alkynyl, reagents for reduction of N0₂ must be selected so as to selectively reduce NO₂ without affecting the R^{4A} group. It may be more suitable that R^{4A} in formula (V) or (VI) is an N-protecting group, that may be removed at an appropriate point in the reaction and replaced by a desired R⁴ group by conventional methods.

Compounds of formulae (IV) and (VI) and the reagents used are commercially available, or can be prepared from commercially available materials using conventional procedures described in the literature, and as illustrated below.

More specifically, compounds of formula (II) in which n=1 and p=2 or n=2 and p=1 are tetrahydroisoquinolines and may be prepared from the corresponding unsaturated compound of formula (VII)

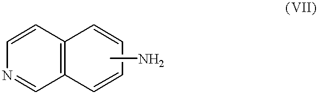

by reaction with a compound R^{4A} where Z is a leaving group such as halogen, especially iodo, or tosylate to obtain an intermediate of formula (VII)

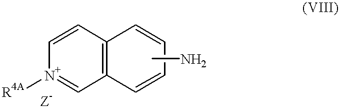

which can be reduced, for example using sodium borohydride, to the compound of formula (II). Alternatively the compound of formula (VIII) can be hydrogenated, for example using hydrogen at 50psi in a solution of acetic/sulphuric acid with a platinum oxide catalyst.

Another route is from a precursor of formula (IX)

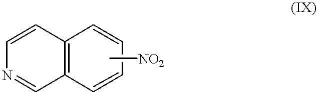

which can be reacted with R^{4A}Z, preferably as a tosylate, to obtain the intermediate of formula (X)

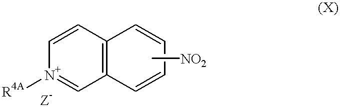

which can then be hydrogenated under the conditions previously described to prepare the compound of formula (II).

When R^{4A} is hydrogen, the compound of formula (II) can be obtained by direct hydrogenation of the compounds of formula (VII) or (IX), using the reagents already described. The NH may be protected conventionally, for example by making R^{4A} t-butoxycarbonyl, prior to formation of the carboxamide, and then deprotected under standard conditions, for example using trifluoroacetic acid/methylene chloride.

Compounds of formulae (VII) and (IX) and the reagents used are commercially available, or can be prepared from commercially available materials using conventional procedures described in the literature.

Compounds of formula (II) in which n=1 and p=1 are amino-dihydroisoindolines. Such compounds may be prepared from compounds of formula (XI)

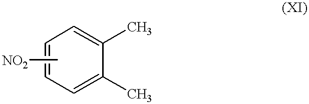

by forming a leaving group, such as bromo, on the methyl groups, and reacting with an amine R^{4A}NH₂ to form the saturated heterocyclic ring, followed by reduction of the nitro group. For example, the compound of formula (XII)

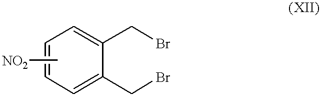

may be formed by refluxing the compound of formula (XI) with N-bromosuccinimide/carbon tetrachloride in the presence of a light source and/or a radical initiator such as t-butyl perbenzoate. The product (XII) can be reacted with R^{4A}NH₂ in methylene dichloride to obtain the compound of formula (XIII)

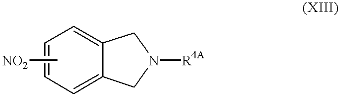

This can be converted to an aminoisoindoline of formula (II) by reduction with hydrogen and a palladium catalyst in ethanol. This route is based on the procedure disclosed in U.S. Pat. No. 5,436,250.

Alternative routes to dihydroisoindolines of formula (II) via a compound of formula (VI) where n=1 and p=1 can be found in Wätjen et al, Biomed.Lett. 1994, 4(2), 371 and Knefeli et al, Arch.Pharm. 1989, 322, 419.

Compounds of formula (II) in which (n+p)=4 are amino-tetrahydrobenzazepines. Such compounds may be prepared from a compound of formula (XIV)

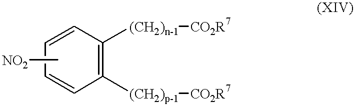

where R⁷ is C₁₋₄alkyl, typically methyl or ethyl, which is reacted with diborane in a suitable solvent such as tetrahydrofuran to give a compound of formula (XV)

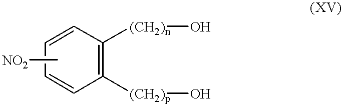

Further reaction with methanesulfonyl chloride in pyridine gives a compound of formula (XVI)

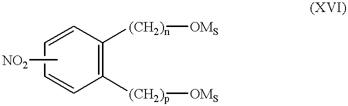

which is a compound of formula (IV) in which X is methanesulfonyl (OMs). This can be reacted with R^{4A}NH₂ in a solvent such as dimethylformamide to obtain a compound of formula (V) with the appropriate n/p values for an amino-tetrahydrobenzazepine.

In this reaction R^{4A} is suitably a protecting group such as benzyl which is easily replaceable by desired R⁴ groups. Further reaction with hydrogen and a palladium catalyst in acetic acid converts the NO₂ group to NH₂ and results in a compound of formula (II). If the R^{4A} group in formula (V) is benzyl then the corresponding formula (II) compound will contain a R⁴ hydrogen group, which can be used as a starting point for additional R⁴ groups by conventional interconversions. This reaction scheme is based on the disclosure of EP-A-0002624 to which reference is directed, and which specifically discloses preparation of aminotetrahydrobenzazepines of formula (II) in which n=1 and p=3 (or vice versa), or as described by R. M. DeMarinis et al, J. Med. Chem., 1984, 27, 918 for n=p=2.

Alternative routes to benzazepines of formula (II) in which n=1 and p=3 can be found in A.I. Meyers et al., Tetrahedron 1993, 49, 1807.”

Compounds of formula (III) can be prepared by further substitution of commercially available benzoic acid derivatives using conventional procedures, by analogy with the procedures set out in the Preparations and Procedures below. Suitable starting materials are 2,4-dimethoxy benzoic acid, 2-methoxy 4-amino benzoic acid and 2-methoxy 4-chloro benzoic acid.

The preparation of compounds of this invention is further illustrated by the following Examples. The Descriptions and Preparations that precede the Examples show the synthesis of the precursor compounds of Formulae (II) and (III) which are coupled in the Examples to produce the novel compounds of this invention. The subsequent Procedures show the synthesis of further compounds of Formulae (II) and (III) which may be coupled with compounds from the Descriptions and Preparations to obtain additional compounds within the scope of this invention. The utility of compounds of this invention is shown by the Pharmacological Data that follow the Examples.

Description 1

N-2-(4-Nitrophenyl)ethyl-trifluoroacetamide

A solution of trifluoroacetic anhydride (10.6ml) in dichloromethane (100ml) was added dropwise to a stirred solution of 2,6-lutidine (17.44ml) and 4-nitrophenethylamine hydrochloride (15.2g; 75 mmol) at 0° C. The mixture was stirred at 25° C. overnight under argon and then washed with dilute citric acid (x2), brine and dried over Na₂SO₄. The material in the organic phase gave the title compound as a pale yellow solid (19.04g).

Description 2

7-Nitro-1,2,3,4-tetrahydro-2-trifluoroacetyl-isoquinoline

The nitro compound D1 (2.26g; 9.15 mmol) and paraformaldehyde (0.45g; 14.4 mmol) in acetic acid (10ml) and conc. H₂SO₄ (15ml ) were stirred at 25° C. for 20h according to the procedure of G.E. Stokker., Tet. Lett., 1996, 37, 5453. Work up afforded the title compound as a white solid (2.17g).

¹H NMR (CDCl₃) δ: 3.10 (2H, m), 3.92 (2H, m), 4.85+4.92 (2H, 2xs), 7.38 (1H, t), 8.10 (2H, m); ^{m}/z (EI): 274 (M⁺)

Description 3

7-Nitro-1,2,3,4-tetrahydroisoquinoline

The trifluoroacetamide D2 (17.22g; 63 mmol) was hydrolysed at room temperature using a solution of potassium carbonate (46.6g) in 10% aqueous methanol (660ml). Work-up with dichloromethane gave the title compound (11g).

Description 4

2-Methyl-7-nitro-1,2,3,4-tetrahydroisoquinoline

The amine D3 (2.08g; 11.7 mmol) was treated with 88% formic acid (3.45ml) and 37% aqueous formaldehyde (5.88ml) at 80° C. for 2h according to the procedure of G.M. Carrera and D.S. Garvey, J. Het. Chem., 1992, 29, 847. Basification with 10% sodium hydroxide followed by work-up with ethyl acetate afforded an orange gum(2.3g). Chromatography on Kiesegel 60 in 0-3% methanol—ethyl acetate gave the title compound as an orange solid (1.7g).

^{m}/z (CI): 193 (MH⁺).

Description 5

7-Amino-2-methyl-1,2,3,4-tetrahydroisoquinoline

The 7-nitro compound D4 (0.25g; 1.3 mmol) in methanol (40ml) was hydrogenated over 10% palladium on carbon (100mg) at atmospheric pressure overnight. The catalyst was removed by filtration through a pad of Kieselguhr and evaporation in vacuo gave the title compound as a white solid (213mg).

^{m}/z (CI): 163 (MH⁺)

Description 6

5-Amino-2-methylisoquinolinium iodide

To a solution of 5-aminoisoquinoline (14.4g, 100mmol) in acetone (300 ml) was added iodomethane (14.4ml). The solution was briefly stirred and then allowed to stand for 2h. The yellow precipitate was then filtered, washed with acetone and dried to afford the title compound as a yellow solid (18.8g).

Description 7

5-Amino-2-methyl-1,2,3,4-tetrahydroisoquinoline

To an ice cold solution of 5-amino-2-methylisoquinolinium iodide (18.8g, 65mmol) in methanol (1.5L) and water (60ml) was added sodium borohydride (17.8g, 0.47mol) portionwise over 2h. The mixture was then allowed to stir at room temperature for 18h before concentration in vacuo and partitioning of the residue between water and dichloromethane. The organic layer was dried over sodium sulfate and concentrated in vacuo to afford the title compound (8.87g).

Description 8

5-Amino-1,2,3,4-tetrahydroisoquinoline

A solution of 5-aminoisoquinoline (10g, 69mmol) in glacial acetic acid (150ml) and concentrated sulfuric acid (1ml) was hydrogenated over platinum oxide (1g) at 55psi for 20h. The acetic acid was then removed in vacuo and the residue treated with saturated aqueous potassium carbonate (100ml) and extracted with dichloromethane. The organic layer was dried over sodium sulfate and concentrated in vacuo to afford the title compound (6.45g).

Description 9

5-Amino-2-(tert-butoxycarbonyl)-1,2,3,4-tetrahydroisoquinoline

An ice cold solution of 5-amino-1,2,3,4-tetrahydroisoquinoline (6.45g, 44mmol) in 1,4-dioxane (250ml) was treated with 3M sodium hydroxide (14.7ml, 44mmol), and di-tert-butyl-dicarbonate (9.57ml, 44mmol) and the solution was stirred at room temperature overnight. The reaction mixture was then poured into water (400ml) and extracted with ether. The organic phases were dried over sodium sulfate and concentrated in vacuo to afford a brown oil which solidified on standing and was recrystallised from ethanol/petrol to afford the title compound as an off white crystalline solid (5.1g).

Description 10

5-Amino-2-[2-(t-butyldimethylsilyloxy)ethyl]-1,2,3,4-tetrahydroisoquinoline.

¹H NMR (CDCl₃) δ: 0.10 (6H, s), 0.90 (9H, s), 2.85 (2H, t, J=6 Hz), 3.13 (2H, t, J=5 Hz), 3.41 (2H, t, J=6 Hz), 4.14 (2H, t, J=5 Hz), 4.20 (2H, s), 6.49 (1H, d, J=8 Hz), 6.54 (1H, d, J=8 Hz), 7.04 (1H, d, J=8 Hz).

Description 11

8-Amino-2-methyl-2,3,4,5-tetrahydro-1H-benzo[c]azepine

Benzazepin-1-one was prepared according to the procedure of A.I.Meyers and R.Hutchings, Tetrahedron 1993, 49, 1807-20 and nitrated according to the procedure of H.Maeda et al., Chem. Pharm. Bull., 1988, 36, 190-201. Standard reduction of the lactam with borane in THF followed by N-methylation and hydrogenolysis in a manner similar to that described in Descriptions 4 and 5 gave the title compound.

¹H NMR (CDCl₃) δ: 1.72 (2H, m), 2.30 (3H, s), 2.76 (2H, m), 2.98 (2H, m), 3.50 (2H, br, ex), 3.71 (2H, s), 6.44-6.55 (2H, m), 6.90 (1H, d, J=8 Hz).

Preparation 1

5-Bromo-2,4-dimethoxybenzoic acid

To a solution of 2,4-dimethoxybenzoic acid (4.0g, 0.022mol ) in chloroform (60ml) was added bromine (1.13ml, 0.022mol) in chloroform (20ml) dropwise. After stirring overnight at room temperature the precipitate was filtered off and dried to afford the title compound as a white solid (2.87g).

Preparation 2

5-Bromo-4-iso-propyl-2-methoxybenzoic acid

To a solution of 2-methoxy-4-iso-propyl benzoic acid (7.0g, 36.0 mmol) in chloroform (100 ml) was added bromine (1.86 ml) in chloroform (20 ml) dropwise. The reaction was stirred at room temperature overnight. Evaporation in vacuo afforded an oil (9.27g).

^{m}/z (CI): 275, 273 (MH⁺; 70%).

Preparation 3

Methyl-5-bromo-4-iso-propyl-2-methoxy benzoate

5-Bromo-4-iso-propyl-2-methoxybenzoic acid (9.268g 34.0 mmol) was dissolved in ethanol (250 ml) and conc. H₂SO₄ (2 ml) added. The mixture was refluxed for 5h and concentrated in vacuo. Residual material was taken up into ethyl acetate and water, and the organic layer, dried (MgSO₄). Concentration in vacuo afforded an oil, which was purified by Biotage Column Chromatography on silica gel using 10% ether in hexane. An oil (5.5g) was obtained.

Preparation 4

2,4-Dimethoxy-5-trifluoromethylbenzoic acid

2,4-Dimethoxy-5-bromobenzoic acid methyl ester (1.5g; 5.4 mmol) in DMF (25ml) and toluene (8ml) under argon was treated with potassium trifluoroacetate (1.53g; 10.1 mmol) and copper (I) iodide (2.1g, 10.9 mmol). The mixture was heated to 170° C. with removal of water (Dean/Stark), and then at 155° C. overnight. The mixture was allowed to cool, poured into ether and water and filtered through Kieselguhr. The organic layer was dried (Na₂SO₄) and concentrated in vacuo to give a brown solid. Chromatography on Kieselgel 60 with 1:1 ether/petrol gave a white solid (1.03g) which was hydrolised in 1:1 methanolic: aqueous NaOH (50ml) at 50° C. Work-up gave the title compound as a white solid (1g).

Preparation 5a

Methyl 2-methoxy-5-cyano-4-iso-propylbenzoate

Copper (I) cyanide (550mg, 6mmol) was added to a solution of methyl 2-methoxy-5-bromro4-iso-propylbenzoate (861mg) in N-methyl-2-pyrolidinone (30ml). The mixture was stirred under argon and boiled under reflux for 4h. The mixture was cooled, poured into excess ice/water and ethyl acetate and filtered. The organic phase was separated, washed with water, brine and dried(MgSO₄). Evaporation gave a crude brown solid which was purified by chromatography on silica gel eluting with ethyl acetate/n-hexane (1:4). The product was obtained as a white solid (523mg).

¹H NMR (250MHz, CDCl₃) δ: 1•33 (6H, d, J=7Hz), 3•38 (1H, sep, J=7Hz), 3•89 (3H, s), 3•98 (3H, s), 6•91 (1H, s), 8•08 (1H, s); ^{m}/z (API⁺): 234 (MH⁺, 30%).

Preparation 5b

2-Methoxy-5-cyano-4-iso-propylbenzoic acid

2N NaOH (1•25ml) was added to a solution of the methyl ester 5a (490mg) in methanol (10ml). The solution was stirred overnight at room temperature. The solution was then diluted with water, concentrated in vacuo and washed with ethyl acetate. The aqueous phase was then acidified with 2N HCl and extracted with ethyl acetate. The extract was washed with brine, dried (MgSO4) and evaporated to dryness giving the product as a white solid (418mg).

¹H NMR (250MHz, CDCl₃) 67 : 1•35 (6H, d, J=7Hz), 3•43 (1H, sep, J=7Hz), 4•14 (3H,s), 7•00 (1H, s), 8•41 (1H, s); ^{m}/z (API⁺): 220 (MH⁺, 100%).

Preparation 6

(a) Ethyl 2-ethoxy-4-iso-propyl-5-cyanobenzoate

Ethyl 2-ethoxy4-iso-propyl-5-bromobenzoate (1•2g, 3•8mmol) was treated with copper (I) cyanide (682mg, 7•6 m.mol) in N-methyl-2-pyrrolidinone (40ml) as described in Preparation 5 to give the title compound as an oil (400mg).

¹H NMR (250MHz, CDCl₃) 67 : 1.12 (6H, d, J=7Hz), 1.30 (3H, t, J=7Hz), 1.84 (3H, t, J=7Hz), 3.17 (1H, sep, J=7Hz), 3.99 (2H, q, J=9Hz), 4.16 (2H, q, J=7Hz), 6.69 (1H, s), 7.86 (1H, s); ^{m}/z (API⁺): 262 (MH⁺,100%).

(b ) 2-Ethoxy-4-iso-propyl-5-cyanobenzoic acid

The ester 6a (370mg, 1.41mmol) was dissolved in methanol (5ml) and over a 24h period IN NaOH (2.1ml, 2.1mmol) was added. The solution was concentrated under vacuum, diluted with water and washed with ethyl acetate. The aqueous phase was acidified with 2N HCl and extracted with ethyl acetate. The extract was washed with brine, dried (Mg SO₄) and evaporated to give the desired acid (306 mg).

¹H NMR (250MHz CDCl₃) 67 : 1.39 (3H, d, J=7Hz), 1.66 (3H, t, J=7Hz), 3.47 (1H, sep, J=7Hz), 4.46 (2H, q, J=7Hz), 7.03 (1H, s), 8.47 (1H, s);

^{m}/z (API⁺): 234 (MH⁺,100%).

Preparation 7

(a) Methyl 2-methoxy-4-iso-propyl-5-pentafluoroethylbenzoate

Methyl 2-methoxy-4-iso-propyl-5-iodobenzoate (334 mg, 1mmol) was dissolved in a mixture of toluene (10ml) and DMF (10ml). Potassium pentafluoropropionate (300mg, 1.5mmol) and copper (I) iodide (380mg, 2mmol) was added and the mixture heated in an oil bath at 145° C. A Dean and Stark apparatus was used to collect distilled toluene and approximately 8 ml were removed. The oil bath temperature was then raised to 160° C. and maintained at that temperature for 5h. The mixture was cooled, filtered and the filltrate diluted with ethyl acetate and water. The organic layer was washed with water, brine and dried (Mg SO₄). Evaporation gave the desired product as a yellow solid, (335mg).

^{m}/z (API⁺): 327 (100%, MH⁺).

(b) 2-Methoxy-4-iso-propyl-5-pentafluoroethylbenzoic acid

1 N NaOH (1.25ml) was added to a solution of 7a (400mg, 1.22mmol). The solution was stirred overnight at room temperature, diluted with water and concentrated in vacuo. Acidification with 2N HCl and work-up with ethyl acetate gave the title compound as an off white solid (232 mg).

¹H NMR (250MHz, CDCl₃), 1.25 (6H, d, J=7Hz), 3.39 (1H, sep, J=7Hz), 4.10 (3H, s), 7.06 (1H, br.s), 8.32 (1H, s); ^{m}/z (API⁺): 313 (100%, MH⁺).

Preparation 8

5-Methoxy-l-indanone-6-carboxylic acid

The compound was prepared from 5-methoxyindan-6-carboxylic acid [made by the procedure of Ciba-Geigy in EP 0 020 301 (1981)] by oxidation with chromium trioxide-acetic anhydride according to the procedure of D.S. Fullerton and C.M. Chen, Syn. Commun., 1976, 6, 217.

Preparation 9

4-Ethoxy-2-methoxy-5-methylsulfonylbenzoic acid

4-Ethoxy-2-methoxy-5-chlorosulfonyl benzoic acid. was prepared in 49% yield using the procedure of M.W. Harrold et al., J. Med. Chem., 1989, 32 874. This was used according to the method of R. W. Brown, J. Org. Chem., 1991, 56, 4974, to the title compound in 19% yield.

¹H NMR (DMSO) 67 : 1.30 (3H, t), 3.10 (3H, s), 3.83 (3H, s), 4.24 (2H, q), 6.73 (1H,s), 8.07 (1H, s).

Preparation 10

4-iso-Propyl-2-methoxy-5-methylsulfonylbenzoic acid

This was prepared in a similar manner to the procedure of C. Hansch, B. Schmidhalter, F. Reiter, W. Saltonstall . J. Org. Chem., 1956, 21, 265 to afford the intermediate 5-chlorosulfonyl-4-isopropyl-2-methoxybenzoic acid which was converted into the title compound using the method of Preparation 9.

¹H NMR (DMSO) 67 : 1.30 (6H, d), 3.21 (3H, s), 3.80 (1H, m), 3.94 (3H, s), 7.26 (1H,s), 8.19 (1H,s).

Preparation 11

2,4-Dimethoxy-5-methylsulfonylbenzoic acid

5—Chlorosulfonyl-2,4-dimethoxybenzoic acid was prepared according to the method described in Preparation 9 in an 87% yield. This was used immediately to give the title compound in 62% yield.

¹H NMR (DMSO) 67 : 3.17 (3H, s), 3.94 (3H, s), 4.03 (3H, s), 6.84 (1H, s), 8.16 (1H,s).

Preparation 12

2-Methoxy4-methyl-5-methylsulfonylbenzoic acid

The title compound was made in a similar manner to that described in Preparation 9 in 39% yield.

¹H NMR (DMSO) δ: 2.64 (3H, s), 3.16 (3H, s), 3.88 (3H, s), 7.20 (1H, s), 8.17 (1H, s).

Preparation 13

4-Ethyl-2-ethoxy-5-methylsulfonylbenzoic acid

To a cooled, stirred solution of chlorosulfonic acid (9ml), 2-ethoxy-4-ethylbenzoic acid (1.55g, 7.99 mmol) was added slowly over 5 min, to limit the exotherm seen. The reaction mixture was then stirred at room temperature for 7h and poured carefully onto cracked ice. The pale yellow solid which precipitated was filtered and dried in vacuo to give 5-chlorosulfonyl-2-ethoxy-4-ethylbenzoic acid in 79% yield. This was used in a similar procedure to the method of Preparation 9 to give the title compound 52% yield.

¹H NMR (d⁶ DMSO) δ: 1.06 (3H, t), 1.15 (3H, t), 2.80 (2H, q), 3.00 (3H, s), 4.03 (2H, q), 6.99 (1H,s), 7.97 (1H,s).

Preparation 14

2,4-Diethoxy-5-trifluoromethylbenzoic acid

The title compound was prepared in 80% overall yield from 5-bromo-2,4-diethoxybenzoic acid methyl ester using potassium trifluoroacetate and copper (I) iodide followed by conventional hydrolysis in a manner similar to that of Procedure 41.

^{m}/z (APT⁺): 279 (MH⁺; 100%).

Preparation 15

2-Ethoxy-4-ethyl-5-fluorobenzoic acid

a) Methyl 2-Ethoxy-4-ethyl-5-fluorobenzoate

Methyl 2-ethoxy4-ethylbenzoate (220mg) was dissolved in dichloromethane (2ml) and added to a stirred solution of xenon difluoride (350mg) in dichloromethane (2ml) under argon and with cooling in ice. After ca 1 h. the ice bath was removed and the solution stirred at room temperature for 3 hours. The reaction mixture was diluted with aqueous sodium bicarbonate and the organic phase separated, washed with brine and dried (MgSO₄). Evaporation of solvent gave an oil which was chromatographed on silica eluting with ethyl acetate/hexane (initially 1:8 then 1:4). The fluoro compound was obtained as an oil (65mg).

^{m}/z (API⁺) 227 (MH⁺,100%)

b) 2-Ethoxy-4-ethyl-5-fluorobenzoic acid

The methyl ester prepared above (65mg) was dissolved in methanol (2ml) and 1M NaOH (0.34ml) added. The solution was left for two days at room temperature, diluted with water and concentrated in vacuo. The aqueous mixture was washed with ethyl acetate, and then acidified with 2M HCl and extracted with ethyl acetate. The extract was washed with brine, dried (MgSO₄) and evaporated in vacuo. Trituration with n-hexane gave the acid as a white solid (30mg).

¹H NMR (250 MHz, CDCl₃) 67 : 1.25 (3H, t, J=7.5 Hz), 1.55 (3H, t, J=7 Hz), 2.72 (2H, m), 4.35 (2H, m), 6.85 (1H, d, J=6 Hz), 7.80 (1H, d, J=10 Hz).

Preparation 16

2,4-Diethoxybenzonitrile

To a solution of 2,4-difluorobenzonitrile (20g, 0.14mol) in dry THF under argon at −78° C. was added potassium ethoxide (26.5g, 0.32mol). The cooling bath was removed and the solution stirred overnight at 25° C. The reaction mixture was partitioned between ether and water. The organic phase was washed with brine, dried (Na₂SO₄) and concentrated in vacuo to afford the title compound as a white solid (26.25g, 96%).

^{m}/z (API⁺): 192 (MH; 100%).

Preparation 17

2,4-Diethoxy-5-bromobenzonitrile

To a solution of 2,4-diethoxybenzonitrile (26.25g, 0.14mol) in chloroform was added a solution of bromine (7.09ml, 0.14mol) in chloroform (75ml). The solution was stirred overnight at 25° C. then concentrated in vacuo to afford the title compound as a yellow solid (36.6g, 99%).

^{m}/z , (API⁺): 272 (MH⁺; 90%).

Preparation 18

2,4-Diethoxy-5-bromobenzaldehyde

To a solution of 2,4-diethoxy-5-bromobenzonitrile (35g, 0.13mol) in dry toluene (700ml) under argon was added DIBAL (86ml of a 1.5M solution in toluene 0.13mol) dropwise. The reaction was then stirred at 25° C. for 4h. 2M HCl (150ml) was then added carefully followed by vigorous stirring for 30 min. After dilution with water (500ml), the two phases were separated and the aqueous layer extracted with diethyl ether (2×150ml). The combined organic phases were then concentrated in vacuo to afford the title compound as a yellow oil (34.1g).

¹H NMR (CDCl₃) δ: 1.46 (6H, m), 4.09 (4H, m), 6.43 (1H,s), 8.00 (1H,s), 10.27 (1H,s).

Preparation 19

2,4-Diethoxy-5-bromobenzylalcohol

To 2,4-diethoxy-5-bromobenzaldehyde (34.1g, 0.13mol) in toluene (1l) at −78° C. under argon was added DIBAL (92ml of a 1.5M solution in toluene). The mixture was then allowed to warm up to 25° C. over 2h. Water (400ml) was gradually added with stirring. The layers were then separated, the organic phase filtered and concentated in vacuo to afford the title compound (34g, 99%).

¹H NMR (CDCl₃) δ: 1.44 (6H, m), 4.05 (4H, m), 4.60 (2H, s), 6.46 (1H, s), 7.40 (1H,s).

Preparation 20

2,4-Diethoxy-5-bromobenzylalcohol-tert-butyldimethylsilyl ether

To a solution of 2,4-diethoxy-5-bromobenzyl alcohol (34g, 0.12mol) in dichloromethane (500ml) was added triethylamine (40ml) followed by tert-butyldimethylsilylchloride (20.76g, 0.14 mol). The solution was stirred overnight at 25° C., diluted with dichloro-methane (250ml), washed with saturated aq. NaHCO₃, dried (Na₂SO₄) and concentrated in vacuo. The residue was chromatographed on silica gel, using 10% diethyl ether: 60-80° pet. ether to afford the title compound as a white solid (25.88g, 54%).

¹H NMR (CDCl₃) δ: 0.11 (6H, s), 0.94 (9H, s), 1.42 (6H, m), 4.04 (4H, m), 4.64 (2H, s), 6.41 (1H, s), 7.53 (1H, s).

Preparation 21

2,4-Diethoxy-5-trifluoroacetyl-benzylalcohol-tert-butyldimethylsilyl ether

To a solution of 2,4-diethoxy-5-bromobenzylalcohol-tert-butyldimethylsilyl ether (4g, 10.3mmol) in dry THF under argon at −78° C. was added n-butyllithium (4.12ml of a 2.5M solution in hexanes, 10.3mmol) dropwise over 2 min. After 30 min at −78° C., N,N-diethylamino-trifluoroacetamide (1.6ml, 11.3mmol) was added dropwise over 1 min. Stirring was continued for 1h then satd. ammonium chloride (30ml) was added at −78° C. The reaction mixture was then allowed to warm up to room temperature and partitioned between diethyl ether and water. The organic phase was washed with brine, dried (Na₂SO₄) and concentrated in vacuo. The residue was filtered through a short pad of silica gel eluting with 20% diethyl ether: 60-80° C. petroleum ether to afford the title compound as a white solid (3.62g, 87%).

¹H NMR (CDCl₃) δ: 0.10 (6H, s), 0.94 (9H, s), 1.43 (6H, m), 4.10 (4H, m), 4.65 (2H, s), 6.43 (1H, s), 7.89 (1H, s).

The following Preparations 22 to 28 were prepared in a similar manner to those for Preparations 2, 3 16 to 21 and Procedure 10 described herein.

Preparation 22

Methyl 2,4-diethoxy-5-acetyl-benzoate

¹H NMR (CDCl₃) δ: 1.40 (6H, m), 2.60 (3H, s), 3.85 (3H, s), 4.17 (4H, m), 6.41 (1H,s), 8.42 (1H, s).

Preparation 23

2,4-Diethoxy-5-acetylbenzoic acid

¹H NMR (DMSO-d₆) δ: 1.54 (3H, t, J=7 Hz), 1.62 (3H, t, J=7 Hz), 2.61 (3H, s), 4.41 (4H, m), 6.85 (1H, s), 8.30 (1H, s).

Preparation 24

2-Methoxy-4-iso-propyloxy-5-bromobenzyl alcohol

¹H NMR (CDCl₃) δ: 1.38 (6H, d, J=7 Hz), 2.10 (1H, t, J=7 Hz), 3.84 (3H, s), 4.53 (3H, m), 6.50 (1H,s), 7.42 (1H, s).

Preparation 25

2-Methoxy-4-iso-propyloxy-5-bromobenzyl alcohol-tert-butyldimethylsilyl ether

¹H NMR (CDCl₃) δ: 0.02 (6H, s), 0.91 (9H, s), 1.33 (6H, d, J=7 Hz), 3.77 (3H, s), 4.49 (1H, m), 4.64 (2H, s), 6.45 (1H, s), 7.53 (1H, s).

Preparation 26

2-Methoxy-4-iso-propyloxy-5-trifluoroacetylbenzyl alcohol-tert-butyldimethysilyl ether

¹H NMR (CDCl₃) δ: 0.01 (6H, s), 0.93 (9H, s), 1.18 (6H, d, J 7 Hz), 3.77 (3H, s), 4.64 (3H, m), 6.39 (1H, s), 7.86 (1H, s).

Preparation 27

2-Methoxy-4-iso-propyloxy-5-trifluoroacetylbenzyl alcohol-methyl ether

¹H NMR (CDCl₃) δ: (6H, d, J=7 Hz), 3.39 (3H, s), 3.91 (3H, s), 4.41 (2H, s), 4.70 (1H, m), 6.42 (1H, s), 7.73 (1H, s).

Preparation 28

2-Methoxy-4-iso-propyloxy-5-trifluoroacetylbenzoic acid

^{m}/z (API⁺): 307 (MH⁺; 80%).

Preparation 29

2,4 Diethoxy-5-pivaloylbenzoic acid

Prepared from 5-bromo-2,4-diethoxybenzyl alcohol in a manner similar to that described in Procedures 37 to 40.

^{m}/z (API⁺): 295.2 (MH⁺; 100%).

Preparation 30

2-Methoxy-4-iso-propyl-5-trifluoromethylsulfonylbenzoic acid

Prepared in a manner similar to that described in Procedures 35 and 36 using copper (1) trifluoromethanethiolate followed by oxidation with permanganate.

¹H NMR (250 MHz CDCl₃) δ: 1.33 (6H, d, J=7 Hz), 4.00 (1H, m, J=7 Hz), 4.17 (3H, s), 7.18 (1H, s), 8.83 (1H, s).

Preparation 31

5—Chloro-2-methoxy-4-iso-propyloxybenzoic acid

Prepared in a manner similar to that described in Procedure 2.

^{m}/z (API⁺): 247.1, 245.1 (MH⁺; 30%, 100%).

Preparation 32

5—Cyano-2-methoxy-4-iso-propyloxybenzoic acid

Prepared in a manner similar to that described in Preparation 5.

¹H NMR (250 MHz CDCl₃) δ:1.46 (6H, d, J=6 Hz), 4.10 (3H, s), 4.75 (1H, m, J=6 Hz), 6.52 (1H, s), 8.37 (1H, s).

Preparation 33

5—Cyano-4-ethyl-2-n-propyloxybenzoic acid

Prepared in a manner similar to that described in Preparation 5.

^{m}/z (API⁺): 234.1 (MH⁺; 50%).

Preparation 34

5-Acetyl-2-methoxy-4-iso-propyloxybenzoic acid

Prepared in a manner similar to that described in Procedures 37 to 40.

^{m}/z (API⁺): 253.1 (MH⁺; 50%).

Preparation 35

4-Ethyl-2-methoxy-5-(N,N-dimethylaminocarbonyl)benzoic acid

¹H NMR (250 MHz CDCl₃) δ: 1.25 (3H, t, J=7.5 Hz), 2.71 (2H, d, J=7.5 Hz), 2.86 (3H, s), 3.13 (3H, s), 4.10 (3H, s), 6.94 (1H, s), 7.98 (1H, s).

Preparation 36

3-Bromo-4-ethoxybenzoic acid

The title compound was prepared from 4-ethoxybenzoic acid in a manner similar to that of Preparation 1.

¹H NMR (DMSO-D₆) δ: 1.45 (3H, t, J=7 Hz), 4.26 (2H q, J=7 Hz), 7.26 (1H, d, J=9 Hz), 7.98 (1H, dd, J=2,9 Hz), 8.12 (1H, d, J=2 Hz)

Preparation 37

4-Methoxy-3-trifluoromethylbenzoic acid

The title compound was prepared from 3-bromo4-methoxybenzoic acid and potassium trifluoroacetate in a manner similat to that of Preparation 14.

¹H NMR (DMSO-D₆) δ: 3.78 (3H, s), 7.18 (1H, d, J=9 Hz), 7.90 (1H, d, J=2 Hz), 8.00 (1H, dd, J=2, 9 Hz), 12.70-13.10 (1H, br,exchangeable)

Preparation 38

4-Methoxy-3-trifluoromethylbenzoyl chloride

The title compound was prepared from 4-methoxy-3-trifluoromethylbenzoic acid with oxalyl chloride and DMF in chloroform at room temperature [D. Levin, Chem. Br., 1977, 20] followed by evaporation in vacuo.

Preparation 39

3-Bromo 4iso-propylbenzoic acid

The title compound was prepared from 4-iso-propylbenzoic acid.

Preparation 40

3-Acetyl-4-ethoxybenzoic acid

Prepared in a similar manner to that desribed for Procedure 37 to 40.

¹H NMR (250MHz, CDCl₃) δ: 1.53 (3H, t, J=7 Hz), 2.65 (3H, s), 4.23 (2H, q, J=7 Hz), 7.01 (1H, d, J=8 Hz), 8.19 (1H, dd, J=8, 2 Hz), 8•48 (1H, d, J=2 Hz).

Preparation 41

3-Chloro-4-ethoxybenzoic acid

¹H NMR (DMSO-D₆) δ: 1.39 (3H, t, J=7 Hz), 4.20 (2H, q, J=7 Hz), 7.22 (1H, d, J=7 Hz), 7.87 (2H, m).

Procedure 1

4-Azido-5-iodo-2-methoxybenzoic acid

To a solution of 4-amino-5-iodo-2-methoxybenzoic acid (300 mg. 1.02 mmol) in trifluoroacetic acid (4ml) at 5° C., was added sodium nitrite (283 mg, 4.1 mmol) portionwise, and the mixture allowed to stir for 30 min. Sodium azide (200 mg, 3.07 mmol) was then added portionwise and the mixture stirred for a further 30 min at 0° C. The mixture was diluted with water, and a yellow solid precipitated. The solid was filtered, washed with cold water and dried, to afford the title compound (274 mg, 84%).

Procedure 2

5-Dichloro-2-methoxybenzoic acid

To an ice cold solution of 4-chloro-2-methoxybenzoic acid (1.0g, 5.36mmol) in trifluoroacetic acid (7ml) was added N-chloromorpholine (0.67g, 5.5mmol) dropwise, maintaining the internal temperature below 10° C. After stirring overnight at room temperature the trifluoroacetic acid was removed in vacuo and the residue partitioned between ethyl acetate and water. The organic layer was dried over magnesium sulfate, concentrated in vacuo and the residue recrystallised from methanol to afford the title compound as a white solid (200mg).

Procedure 3

5—Chloro-2,4-dimethoxybenzoic acid

The title compound was prepared in an analogous fashion to Procedure 2 from 2,4-dimethoxybenzoic acid (1.3g). Recrystallisation of the crude product from methanol afforded the title compound as a white solid (1.3g).

Procedure 4

(5-Bromo-2-methoxybenzyloxy)-tert-butyldimethylsilane

To a solution of 5-bromo-2-methoxybenzyl alcohol (1.0 g, 4.6 mmol) and imidazole (470 mg, 7.01 mmol) in DMF (15ml) was added tert-butyldimethylsilyl chloride (1.04 g, 6.91 mmol). The mixture was allowed to stir for 4h, poured onto water (100ml) and extracted with ether (3×30ml). The combined organic phases were washed with water (50ml), brine (50ml), dried over sodium sulfate and evaporated under reduced pressure to leave a pale yellow oil. This was purified by chromatography (SiO₂, 5% ether/petrol), to give the title compound (1.46g, 96%) as a colourless oil.

Procedure 5

(5-Trifluoroacetyl-2-methoxybenzyloxy)-tert-butyldimethylsilane

To a solution of 5-bromo-2-methoxybenzyloxy)-tert-butyldimethylsilane (1.0 g, 3.02 mmol) in THF (5ml) at −78° C., was added n-BuLi (2.26 ml of a 1.6 M solution in pentane, 3.62 mmol) dropwise over 10 min. The solution was allowed to stir for a further 1 h at −78° C., to give a bright yellow solution. N,N-diethyltrifluoroacetamide (561 mg, 3.32 mmol) in THF (2 ml) was added dropwise over 30 min, and the solution was stirred for a further 1 h at −78° C. Saturated aqueous ammonium chloride (5 ml) was added and the mixture allowed to warm to room temperature, and extracted with ether (3×10 ml). The combined organic phases were washed with water (10 ml), brine (10 ml), dried over sodium sulfate and evaporated under reduced pressure. The resulting residue was purified by chromatography (SiO₂, 5% ether/petrol) to give the title compound (0.99g, 94%) as a white solid.

Procedure 6

5 (E, Z)-1-[3-(tert-Butyldimethylsilanyloxymethyl)-4-methoxyphenyl]-2,2,2-trifluoroethanone oxime

A mixture of (5-trifluoroacetyl-2-methoxybenzyloxy)-tert-butyldimethylsilane (1.0 g, 2.87 mmol), hydroxylamine hydrochloride (240 mg, 3.44 mmol), pyridine (18 ml) and ethanol (9 ml) were heated at reflux for 4 h. The resulting mixture was evaporated under reduced pressure and the residue purified by chromatography (SiO₂, 20% ether/petrol), to give an approximately 3:2 inseparable isomeric mixture of (E, Z)-1-[3-(tert-butyldimethylsilanyl oxymethyl)-4-methoxyphenyl]-2,2,2-trifluoroethanone oximes (1.02 g, 98%) as a colourless oil.

Procedure 7

(E, Z)-(4-Toluensulfonyl)-1-[3-(tert-butyldimethylsilanyloxymethyl)-4-methoxy phenyl]-2,2,2-trifluoroethanone oxime

To a solution of (E, Z)-1-[3-(tert-butyldimethylsilanyloxymethyl)-4-methoxyphenyl]-2,2,2-trifluoroethanone oximes (1.0 g, 2.75 mmol), triethylamine (340 mg, 3.36 mmol), DMAP (31 mg, 0.25 mmol) in dichloromethane (5ml) at 0° C., was added tosyl chloride (627 mg, 3.29 mmol) portionwise. The mixture was stirred for 1 h at room temperature and then poured onto water (10 ml). The layers were separated, and the aqueous phase extracted with dichloromethane (3×10 ml). The combined organic phases were washed with water (10 ml), dried (Na₂SO₄) and evaporated under reduced pressure. The residue was purified by chromatography (SiO₂, 20% ether/petrol) to give an inseparable mixture of (E, Z)-(4-toluensulfonyl)-1-[3-(tert-butyldimethylsilanyloxymethyl)-4-methoxyphenyl]-2,2,2-trifluoroethanone oximes (1.39 g, 98%) as a colourless oil.

Procedure 8

3-[3-(tert-Butyldimethylsilanyloxymethyl)-4-methoxyphenyl]-3-trifluoromethyldiaziridine

A solution of (E, Z)(4-toluensulfonyl)-1-[3-(tert-butyldimethylsilanyloxymethyl)-4-methoxyphenyl]-2,2,2-trifluoroethanone oximes (517 mg, 1 mmol) in ether (5 ml) was stirred with liquid NH₃ (15 ml) in a bomb for 4 h at room temperature. The mixture was then filtered and the solid washed with ether. The filtrate was evaporated under reduced pressure, and the residue purified by chromatography (SiO₂, 20% ether/petrol) to give the title compound (350 mg, 97%) as a pale yellow oil.

Procedure 9

3-[3-(tert-Butyldimethylsilanyloxymethyl)-4-methoxyphenyl]-3-trifluoromethyl-3H-diazirine

A mixture of 3-[3-(tert-butyldimethylsilanyloxymethyl)4methoxyphenyl]-3-5 trifluoromethyldiaziridine (200 mg, 0.55 mmol) and freshly prepared Ag₂O (255 mg, 1.1 mmol) in ether (3ml) was stirred for 24 h. The solid was filtered, washed with ether, and the filtrate evaporated under reduced pressure. The resulting residue was purified by chromatography (SiO₂, 10% ether/petrol) to give the title compound (187 mg, 94%) as a colourless oil.

Procedure 10

2-Methoxy-5-(3-trifluoromethyl-3H-diazirin-3-yl)benzoic acid

A solution of 3-[3-(tert-butyldimethylsilanyloxymethyl)4-methoxyphenyl]-3-trifluoromethyl-3H-diazirine (150 mg, 0.41 mmol) in methanol (5 ml) was stirred for 20 min with conc. HCl (2 drops). The solution was poured onto saturated aqueous sodium bicarbonate (10 ml) and extracted with dichloromethane (3×5 ml). The combined organic extracts were dried over sodium sulfate and evaporated under reduced pressure. The residue was taken up in dioxane (3 ml) and aqueous potassium hydroxide (2.5 ml of a 0.2M solution), potassium permanganate (98 mg, 0.62 mmol) was added and the mixture stirred for 4 h. The mixture was filtered through a pad of Celite and washed with water. The filtrate was extracted with ether (2×10 ml). The aqueous phase was brought to pH 1, extracted with ether (2×10 ml) and these extracts were dried over sodium sulfate and evaporated under reduced pressure to give the title compound (77 mg, 72%) as an off white solid.

Procedure 11

5—Chloro-2,4-dimethoxybenzoyl chloride

A solution of 5-chloro-2,4-dimethoxybenzoic acid (6.4g) in dichloromethane (250ml) was treated with thionyl chloride (30ml) and the mixture heated at reflux for 18h. Removal of volatile material in vacuo afforded the title compound as a white solid (6.6g).

Procedure 12

5—Chloro-2,4-dimethoxy-N-[2-(tert-butoxycarbonyl)-1,2,3,4-tetrahydroisoquinolin-5-yl)benzamide

To a solution of 5-amino-2-(tert-butoxycarbonyl)-1,2,3,4-tetrahydroisoquinoline (1g, 4mmol) in dichloromethane (30ml) and triethylamine (3ml) was added 5-chloro-2,4-dimethoxybenzoyl chloride (1.03g, 4.4mmol). After stirring at room temperature for 2h the reaction mixture was diluted with dichloromethane (75ml) and washed with saturated aqueous sodium bicarbonate. The organic layer was dried over magnesium sulfate, concentrated in vacuo and the residue recrystallised from ethyl acetate/petrol to afford the title compound as a colourless crystalline solid (1.3g).

Procedure 13

5—Chloro-2-methoxy-4-methylbenzoic acid

The title compound was prepared in an analogous manner to Procedure 2 from 2-methoxy-4-methylbenzoic acid (3.0g, 0.018 mol). Purification of the crude product using a chromatotron (SiO2, 10% ethyl acetate in hexane) afforded the title compound as a white solid (0.40g)

Procedure 14

5-Amino-2-ethyl-1,2,3,4-tetrahydroisoquinoline

The title compound was prepared by treatment of 5-aminoisoquinoline with iodoethane followed by reduction with sodium borohydride using procedures analogous to those described in Description 6 and Description 7.

Procedure 15

5-Amino-2-propyl-1,2,3,4-tetrahydroisoquinoline

The title compound was prepared by treatment of 5-aminoisoquinoline with iodopropane followed by reduction with sodium borohydride using procedures analogous to those described in Description 6 and Description 7.

Procedure 16

4-Benzyloxy-5-chloro-2-methoxybenzoic acid

A solution of chlorine (5.1g) in acetic acid (100ml) was added dropwise to a solution of methyl 4-benzyloxy-2-methoxybenzoate (10g) in acetic acid (40ml) whilst maintaining the temperature at 20-25° C. The mixture was poured into ice-water and extracted with dichloromethane. The organic extract was dried over sodium sulfate and concentrated in vacuo. The resulting crude material was suspended in ethanol (500ml) and treated with 10% aqueous sodium hydroxide (16ml). The mixture was heated at reflux overnight and then concentrated in vacuo. The residue was treated with excess 5M HCl and extracted into dichloromethane. The extract was dried over sodium sulfate and concentrated in vacuo to afford a white solid which was crystallised from ethanol to give the title compound (6.3g).

Procedure 17

4-Hydroxy-2-methoxybenzoic acid methyl ester.

4-Amino-2-methoxy benzoic acid methyl ester (15g, 82.7mmol) was dissolved in sulfuric acid ( 80ml of a 25% solution). The solution was cooled in an ice bath and diazotized with saturated sodium nitrite solution (8.57g, 124mmol) maintaining the temperature below 5° C. The diazonium solution was poured slowly into boiling sulphuric acid (1L of a 3% solution) and the mixture was heated for an additional 5 mins. The mixture was then allowed to cool before being extracted with dichloromethane. The organic extracts were combined, dried over sodium sulfate and concentrated in vacuo to afford a brown solid (9.7g).

Procedure 18

4-Ethoxy-2-methoxy-benzoic acid methyl ester

To a solution of 4-hydroxy-2-methoxybenzoic acid methyl ester (4.17g, 22.mmol) in DMF (50ml) under argon was added potassium carbonate (6.33g, 4.6mmol) followed by iodoethane (7.15g, 4.6mmol). The mixture was then heated to 50° C. under argon for 12h. On cooling the mixture was poured into a large excess of water and extracted with ether. The combined organic extracts were dried over sodium sulfate and concentrated in vacuo to afford the title compound as a brown oil (4.8g).

Procedure 19

5—Chloro-4-ethoxy-2-methoxybenzoic acid

Trifluoroacetic acid (35ml) was cooled in an ice bath. 4-Ethoxy-2-methoxy- benzoic acid methyl ester (4.85g, 23mmol) was then added slowly. N-chloromorpholine (3.64g, 29.9mmol) was then added dropwise maintaining the reaction mixture temperature below 10° C. The ice bath was removed and the mixture stirred under argon for 12h at room temperature. The solvent was then removed in vacuo and the residue taken up in ethyl acetate and washed wth water. The organic layer was dried over sodium sulfate and concentrated in vacuo to afford a brown oil which was triturated with ether and 60/80 petrol. The resulting brown solid was then recrystallised from 60/80 petrol, taken up into ether and washed with sodium hydroxide solution (2M). The organic layer was dried over sodium sulfate and concentrated in vacuo to afford the methyl ester as a pale yellow solid.(0.9g). A mixture of this ester (0.9g, 3.6mmol), methanol (22ml) and sodium hydroxide solution (20ml, 2M) was heated to 70° C. overnight. On cooling the mixture was acidified to pH 6-7 and the solvent was removed in vacuo The residue was taken up in ethanol and the inorganic solid filtered off. The filtrate was concentrated in vacuo to afford the title compound as a pale brown solid (0.44g).

Procedure 20

5-Bromo-2-methoxy-4-methylbenzoic acid

The title compound was prepared in an analogous manner to Preparation 1 from 2-methoxy-4-methylbenzoic acid (3.0g, 0.018mol). Recrystallisation of the crude product from dichloromethane afforded the title compound as a white solid (0.99g).

Procedure 21

[4-(tert-Butyldimethylsilanyloxymethyl)-3-methoxyphenyl]-phenylmethanol

To 2-(tert-Butyldimethylsilanyloxymethyl)-5-bromoanisole (500 mg, 1.51 mmol) in THF (10ml) at −78° C. was added n-BuLi (1.13 ml of a 1.6M solution in pentane, 1.81 mmol) and the mixture allowed to stir for 1 h at −78° C. Benzaldehyde (176 mg, 1.66 mmol) was added and the mixture allowed to warm to room temperature and stirred for 1 h. Water (20 ml) was added and the mixture extracted with ether (3×10 ml). The combined extracts were washed with water (10 ml), brine (10 ml), dried (Na₂SO₄) and evaporated under reduced pressure. The resulting residue was purified by chromatography (SiO₂, 50% ether/petrol) to give the title compound as a colourless oil (303 mg, 56%).

Procedure 22

[5-(tert-Butyldimethylsilanyloxymethyl)-4-methoxyphenyl]-phenylmethanol

The title compound was prepared in an analogous manner to Procedure 21 from 2-(tert-Butyldimethylsilanyloxymethyl)-4-bromoanisole (500 mg, 1.51 mmol). The crude product was purified by chromatography (SiO₂, 50% ether/petrol) to give the title compound as a colourless oil (63%).

Procedure 23

4-Benzoyl-2-methoxybenzoic acid

A solution of 4-(tert-butyldimethylsilanyloxymethyl)-3-methoxyphenyl]-phenylmethanol (200 mg, 0.56 mmol) in THF (5 ml) was stirred with 5N HCl (5ml) for 1 h. The mixture was poured onto saturated aqueous sodium bicarbonate (10 ml) and extracted with ether (3×10 ml). The combined organic extracts were dried (Na₂SO₄), and evaporated under reduced pressure. The residue was taken up in dioxane (4 ml) and aqueous KOH (5.6 ml of a 0.2M solution), potassium permanganate (266 mg, 1.68 mmol) was added and the mixture stirred for 4 h. The mixture was filtered through a pad of Celite and washed with water. The filtrate was extracted with ether (2 ×10 ml) and the aqueous phase was brought to pH 1 and extracted with ether (2 ×10 ml). These extracts were dried over sodium sulfate and evaporated under reduced pressure to afford the title compound as a white foam (102 mg, 71%).

Procedure 24

5 S—Benzoyl-2-methoxybenzoic acid

The title compound was prepared in an analogous manner to Procedure 23 from [5-(tert-butyldimethylsilanyloxymethyl)-4-methoxyphenyl]-phenylmethanol (200mg, 0.56mmol). The title compound was obtained as a white foam (74%)

Procedure 25

3-Nitro-N-methylphthalimide

A solution of 3-nitrophthalimide (Aldrich)(1.78g, 0.01mol) in dry N,N-dimethylformamide (20ml) was added dropwise to a stirred suspension of sodium hydride (0.36g of an 80% dispersion in oil; 0.012mol) in dry N,N-dimethylformamide (10ml) under argon. The mixture was stirred at room temperature for 30min and then treated with iodomethane (0.75ml, 0.012mol). After stirring overnight the reaction was poured into ice-water and extracted with dichloromethane (4×50ml). The combined extracts were washed with water followed by brine, then dried over sodium sulfate and concentrated in vacuo. The residue was treated with water, and the resulting precipitate was removed by filtration and washed with water. After drying in a vacuum dessicator over silica gel the title compound was obtained as a yellow solid (1.64g).

Procedure 26

4-Amino-2,3-dihydro-2-methyl-1H-isoindole

A solution of 3-nitro-N-methylphthalimide (0.58g, 2.8mmol) in dry tetrahydrofuran (10ml) was added dropwise to a stirred suspension of lithium aluminium hydride (0.64g, 16.8mmol) under argon. The mixture was stirred at room temperature for 2h and then heated under gentle reflux for 2.5h. The reaction was quenched by addition of wet diethyl ether followed by a minimum amuont of water. The precipitated aluminium salts were removed by filtration. The filtrate was concentrated in vacuo to give the title compound as a brown oil (400mg).

Procedure 27

6-Amino-3-methyl-2,3,4,5-tetrahydro-1H-3-benzazepine

9-Amino-6-chloro-3-methyl-2,3,4,5-tetrahydro-1H-3-benzazepine, prepared according to R.M. DeMarinis et al, J. Med. Chem., 1984, 27, 918, (0.190g, 0.90 mmol) was dissolved in 10% acetic acid in methanol (50ml)and 10% Pd/C (150mg) added. The mixture was stirred at room temperature, under hydrogen/atmos pressure for 4h, then filtered through Kieselguhr and evaporated in vacuo. Residual material was taken up into dichloromethane and 5% NaHCO₃ solution; the organic layer was washed with brine, dried (Na₂SO₄) and evaporated in vacuo to afford the title compound as a colourless oil (133mg).

Procedure 28

6-Amino-2-(t-butyloxycarbonyl)-1,2,3,4-tetrahydroisoquinoline

A solution of 6-amino-1,2,3,4-tetrahydroisoquinoline (0.74g; 5 mmol) in 1,4-dioxan (50ml) containing 5M-NaOH (1ml) was stirred at 5° C. and treated with di t butyl dicarbonate (1.09g; 5 mmol). After 20 min at room temperature the product was extracted with ethyl acetate and the material in the organic layer gave a brown gum which was chromatographed on Kieselgel 60 in 5% methanol:dichloromethane. The title compound was obtained as a pale gum (0.55g).

¹H NMR (CDCl₃) δ: 1.49 (9H, s), 2.74 (2H, t), 3.60 (4H, br), 4.46 (2H, s), 6.47 (1H, d), 6.55 (1H, dd), 6.90 (1H, d).

Procedure 29

3-tert-butyl-phenylacetate

A mixture of 3-tert-butylphenol (25.25g, 0.1680 mole), acetic anhydride (34.31g, 0.336 mole) and sodium acetate (13.78g, 0.1680 mole) was heated at 100° C. for 2 h. On cooling the mixture was poured into water (200ml) and extracted with ethyl acetate (200ml). The combined organic extracts were dried over sodium sulphate and concentrated in vacuo to afford the acetate compound as an oil (33.33g).

Procedure 30

4-tert-butyl-2-hydroxy acetophenone

A mixture of the acetate of Procedure 29 (33.23g, 0.173 mole) and AlCl₃ (25.61 g, 0.192 mole) was placed in an oil bath preheated to 120° C. and stirred mechanically. Then the oil bath temperature was raised to 165° C. and maintained for 45 min before being allowed to cool to 120° C. Then water was added dropwise into the reaction mixture (4×250ml) to steam distil the product (bath temp 190-200° C.). The distillate was extracted with ether and the combined organic extracts were dried over sodium sulphate and concentrated in vacuo to afford 4-tert-butyl-2-hydroxy acetophenone as an oil (18.05g).

Procedure 31

4-tert-Butyl-2-methoxy acetophenone

A suspension of 4-tert-butyl-2-hydroxy acetophenone (12.65g), potassium carbonate (13.14g) and dimethyl sulphate (8.99ml) in acetone (200ml) was refluxed for 48h. After cooling, the mixture was filtered. The solvent was then removed in vacuo and the residue taken up in dichloromethane and washed with brine. The organic layer was dried over sodium sulphate and concentrated in vacuo to afford a yellow oil (12.05g).

Procedure 32

4-tert-Butyl-2-methoxybenzoic acid

The acetophenone of Procedure 31 (11.0g, 53 mmol) was added to a solution of sodium hydroxide (28.68g), sodium hypochlorite (182ml, 12% w/w) and water (70ml) at 80° C. with stirring. After heating for 1.25h, the mixture was cooled to 0° C. and a solution of sodium metabisulphite (41.1g) in water (170ml) was added. The mixture was stirred for 15 min and then acidified (pH1) with conc. HCl (45ml). Extraction with ethyl acetate gave the title compound as a white solid (8.9g).

¹H NMR (DMSO-d⁶) δ: 1.30 (9H, s), 3.85 (3H, s), 6.96-7.12 (2H, m), 7.60 (1H, d), 12.30-12.60 (1H, br).

Procedure 33

4-n-Butyl-1-2-methoxybenzoic acid

A mixture of 4-bromo-2-methoxybenzoic acid methyl ester (3.0g, 0.0122 mole), lithium chloride (1.56g), tetra butyl tin (4.51g) and bis (triphenyl phosphine palladium (II) chloride (214mg, 0.3 mmol) were heated at 100° C. for 24h. The solvent was then removed under vacuo and the residue taken up in dichloromethane. The black solid was removed by filtration and the filtrate concentrated in vacuo to give a yellow oil. The oil was purified by column chromatography (Biotage) on silica gel using 10% ether in hexane to afford a colourless oil (1.63g). A portion of the foregoing 4-n-butyl-2-methoxybenzoic acid methyl ester (1.50g) was dissolved in methanol (35ml) with sodium hydroxide solution (2N, 30ml). The mixture was allowed to stir at room temperature overnight. Then added dil. HCl until pH-5. The solvent was then removed in vacuo and the residue taken up in ethyl acetate and washed with brine. The organic layer was dried over sodium sulphate and concentrated in vacuo to afford an oil (1.02g).

Procedure 34

4-n-Butyl-2-methoxy-5 chlorobenzoic acid

4-n-Butyl-2-methoxybenzoic acid (0.5g; 2.9 mmol) and N-chloromorpholine (356mg; 2.9 mmol) were treated in a similar manner to that described in Procedure 19 to give the title compound as a white solid (0.4g).

Procedure 35

Methyl-4-iso-propyl-2-methoxy-5-trifluoromethyl benzoate

A mixture of methyl-5-bromo4-iso-propyl-2-methoxy benzoate (5.43g, 0.0189 mole), potassium trifluoroacetate (5.75g, 0.0378 mole) and copper (I) iodide (7.92g, 0.042 mole) in DMF (100ml) and toluene (30ml) were heated at 170° C. under argon to remove water (Dean-Stark Trap) and then heated to 155° C. overnight. On cooling, after concentration in vacuo, the mixture was poured into ether (300ml) and water (300ml). After filtration through Kieselguhr, the organic layer was separated, washed with brine and dried (Na₂SO₄). Concentration in vacuo afforded a brown yellow solid (4.85g).

Procedure 36

4-iso-Propyl-2-methoxy-5-trifluoromethyl benzoic acid

Methyl4-iso-propyl-2-methoxy-5-trifluoromethyl benzoate was dissolved in methanol (100ml), containing sodium hydroxide solution (2N, 100ml). The mixture was allowed to stir at R.T. overnight and then dil. HCl added until pH-5. The solvent was then removed in vacuo and the residue taken up in ethyl acetate and washed with brine. The organic layer was dried over sodium sulphate and concentrated in vacuo to afford a crude solid which was recrystallised with dichloromethane and hexane to give a solid (2.59g).

Procedure 37

5-Pivaloyl-2-methoxy benzoic acid

5-Pivaloyl-2-methoxy benzyl alcohol (1.19g, 5.35 mmol) was dissolved in dioxane (20ml). A solution of KOH (0.449g, 8.025 mmol in water (5ml)) was added followed by KMnO₄ (1.69g, 10.7 mmol). The mixture was allowed to stir at room temperature over the weekend. The solution was filtered through Celite and extracted with ether. The aqueous phase was acidified with dil. HCl and extracted with ether (3×50ml). The organic layer was dried over magnesium sulphate and concentrated in vacuo to afford the title compound as a white solid (1.06g).

Procedure 38

5-Pivaloyl-2-methoxy benzyl alcohol

5-Pivaloyl-2-methoxy benzyl TBDMS ether (1.8g, 5.35 mmol) was dissolved in methanol (30ml); conc. HCl (20 drops) was added and the whole allowed to stir at room temperature for 4h. Saturated NaHCO₃ solution was added and the mixture extracted with ether (2×100ml). The organic layer was dried over sodium sulphate and concentrated in vacuo to afford the title compound as a colourless oil (1.19g).

Procedure 39

5-Pivaloyl-2-methoxy benzyl TBDMS Ether

n-Butyllithium (11.43ml, 0.0183 mole, 1.6M in hexane) was slowly added to a solution of 5-bromo-2-methoxy benzyl TBDMS ether in tetrahydrofuran (30ml) over 45 mins at −78° C. The reaction mixture was maintained under argon at −78° C. for 1h. Then N,O-dimethylhydroxy pivaloyl amide (2.43g, 0.0167 mole) was added dropwise with stirring at −78° C. The resulting mixture was allowed to stir at −78° C. for 2.5h, quenched with NH₄Cl solution and allowed to warm to room temperature. The mixture was extracted with ether (2×50ml), the combined organics were dried (Na₂SO₄) and concentrated in vacuo to give an oil. The oil was purified by Biotage column chromatography on silica gel using 5% ether in hexane to afford the title compound as a colourless oil (2.95g).

Procedure 40

5-Bromo-2-methoxy benzyl TBMS ether

To a solution of 5-bromo-2-methoxy benzyl alcohol (20.87g, 0.096 mole) in dichloromethane (300ml), Et₃N (20.90 ml, 0.15 mole) was added tert-butyldimethylsilyl chloride (15.94g, 0.10 mole) dropwise. The mixture was allowed to stir at room temperature overnight, then water (300ml) was added. The organic layer was washed with brine, dried (Na₂SO₄) and evaporated to give a white solid. The title compound was purified by dry flash column chromatography on silica gel using 20% ether in hexane to give a white solid (20.1g).

Procedure 41

2,4-Dimethoxy-5-trifluoromethylbenzoic acid

2,4-Dimethoxy-5-bromobenzoic acid methyl ester (1.5g; 5.4 mmol) in DMF (25ml) and toluene (8ml) under argon was treated with potassium trifluoroacetate (1.53g; 10.1 mmol) and copper (I) iodide (2.1g, 10.9 mmol). The mixture was heated to 170° C. with removal of water (Dean/Stark), and then at 155° C. overnight. The mixture was allowed to cool, poured into ether and water and filtered through Kieselguhr. The organic layer was dried (Na₂SO₄) and concentrated in vacuo to give a brown solid. Chromatography on Kieselgel 60 with 1:1 ether/petrol gave a white solid (1.03g) which was hydrolised in 1:1 methanolic: aqueous NaOH (50ml) at 50° C. Work-up gave the title compound as a white solid (1g ).

Reference Example 1

N-(1,2,3,4-Tetrahydroisoquinolin-5-yl)-5-bromo-2,4-dimethoxybenzamide, trifluoroacetate

Prepared from the amine of Description 9 and the acid of Preparation 1 using a procedure similar to that of Example 4. The resultant product was deprotected using trifluoroacetic acid in dichloromethane at room temperature for 12h. and evaporation in vacuo gave the title compound.

¹H NMR (400 MHz, CDCl₃+MeOH-d⁴) δ: 2.99 (2H, t, overlapping HOD), 3.48 (2H, t), 4.00 (3H, s), 4.11 (3H, s), 4.34 (2H, s), 6.58 (1H,s), 7.02 (1H, d), 7.27-7.36 (1H, m, overlapping CHCl₃), 7.75 (1H, d), 8.38 (1H,s).

Reference Example 2

N-(1,2,3,4-Tetrahydroisoquinolin-5-yl)-4-tert-butyl-2-methoxybenzamide hydrochloride

¹H NMR (DMSO-d⁶)δ: 1.31 (9H, s), 2.97 (2H, m), 3.37 (2H, m), 3.44 (2H, m), 4.06 (3H, s), 4.31 (2H, m), 7.08 (1H, d, J=9 Hz), 7.16 (2H, m), 7.28 (1H, m), 7.84 (2H, m), 9.55 (2H, br s), 9.83 (1H,s). ^{m}/z (CI): 339 (MH⁺; 80%).

### EXAMPLE 1.

N-(2-Acetyl-1,2,3,4-tetrahydroisoquinolin-5-yl)-5-bromo-2,4-dimethoxybenzamide

Triethylamine (87μL, 0.62mmol) was added to a stirred suspension of N-(1,2,3,4-tetrahydroisoquinolin-5-yl)-5-bromo-2,4-dimethoxybenzamide trifluoroacetate from Reference Example 1 (315mg; 0.62mmol) in dichloromethane (10ml) at 0° C. Acetic anhydride (60μL; 1eq) was added and the mixture stirred at room temperature overnight. The mixture was washed with water, brine and dried followed by evaporation in vacuo. Recrystallisation of the residue from methanol gave the title compound as a white solid (201mg; 75%).

¹H NMR (CDCl₃, 250MHz): [mixture of rotamers in approximate ratio 1.4:1] δ: 2.20 (3H, s), 2.75 and 2.82 (2H, 2x t), 3.72 and 3.92 (2H, 2x t), 3.99 (3H, s), 4.10 (3H, s), 4.66 and 4.78 (2H, 2x s), 6.53 (1H, s), 6.93 and 7.03 (1H, 2x d), 7.28 (1H, m overlapping with CHCl₃), 7.78 and 8.13 (1H, 2x d), 8.48 (1H,s), 9.38 and 9.54 (1H, 2x br.s); ^{m}/z (API⁺): 433 (MH⁺, Br⁷⁹, 100%); 435 (MH⁺, Br⁸¹, 94%).

### EXAMPLE 2.

N-(1,2,3,4-Tetrahydro-2-trifluoroacetyl-isoquinolin-5-yl)-4-t-butyl-2-methoxybenzamide

Made from Reference Example 2 using a procedure similar to that of Example 1.

¹H NMR (250 MHz, CDCl₃) δ: [mixture of rotamers in approx. ratio 1.4:1] 1.38 (9H, s), 2.90 (2H, m), 3.90 and 3.98 (2H, 2x t), 4.08 and 4.09 (3H, 2x s), 4.79 and 4.83 (2H, 2x s), 6.9-7.1 (2H, m), 7.19 (1H, dd), 7.30 (1H t overlapped by CHC₃), 7.93 and 8.10 (1H, 2x d) 8.24 (1H, dd) 9.65 and 9.72 (1H, 2x br.s).

^{m}/z (API⁺): 457 (MNa⁺; 11%), 435 (MH⁺; 100%).

### EXAMPLE 3.

N-(2-Methanesulfonyl-1,2,3,4-tetrahydroisoquinolin-5-yl)-4-t-butyl-2-methoxybenzamide

Made from Reference Example 1 using methanesulfonyl chloride in dichloromethane containing triethylamine using a procedure similar to that of Example 1.

¹H NMR (250 MHz; CDCl₃)δ: 1.37 (9H, s), 2.87 (3H, s), 2.92 (2H, t), 3.63 (2H, t), 4.09 (3H, s), 4.49 (2H, s), 6.93 (1H, d), 7.05 (1H, d), 7.19 (1H, dd), 7.28 (1H, t), 8.05 (1H, d), 8.23 (1H, d), 9.71 (1H, br.s); ^{m}/z (API⁺) 417 (MH⁺; 100%).

### EXAMPLE 4.

N-(2-Methyl-1,2,3,4-tetrahydroisoquinolin-5-yl)-3-methoxynaphthalene-2-carboxamide

3-Methoxy-2-naphthoic acid (202mg; 1mmol), 1-hydroxybenzotriazole (135mg; 1mmol) and 1-(3-dimethylaminopropyl)-3-ethyl carbodiimide hydrochloride (192mg; 1mmol) in dry dimethylformamide (15ml) was stirred at 25° C. for 40 min. The amine of Description 7 (162mg; 1mmol) was added and the mixture kept at 25° C. overnight. Chloroform (50ml) was added and the organic phase washed with water, brine and dried (Na₂SO₄). Evaporation in vacuo followed by trituration with methanol-ether gave the title compound.

^{m}/z (API⁺): 347 (MH⁺; 100%).

The following Examples 5-9 are prepared using methods similar to those described in the above Descriptions, Preparations and Examples.

### EXAMPLE 5.

N-(2-Methyl-1,2,3,4-tetrahydroisoquinolin-5-yl)-6-methoxy-indane-5-carboxamide, hydrochloride

Prepared by coupling 5-amino-2-methyl-1,2,3,4-tetrahydroisoquinoline D7 with 6-methoxy-indane-5-carboxylic acid (see EP-A-0020301—Ciba Geigy).

¹H NMR (CDCl₃) [free base] δ: 2.11 (2H, m), 2.48 (3H, s), 2.83 (8H, m), 3.60 (2H, s), 4.02 (3H, s), 6.83 (1H, d, J=8Hz), 6.91 (1H, s), 7.20 (1H, t, J=8Hz), 8. 15 (2H, m). 9.77 (1H, br.s); ^{m}/z (API⁺): 337 (MH⁺).

### EXAMPLE 6.

N-(2-Methyl-1,2,3,4-tetrahydroisoquinolin-7-yl)-6methoxy-indane-5-carboxamide hydrochloride

Prepared as in Example 5, but starting from 7-amino-2-methyl-1,2,3,4-tetrahydroisoquinoline D5.

¹H NMR (CDCl₃) [free base] δ: 2.11 (2H, m), 2.46 (3H, s), 2.69 (2H, t, J=6Hz), 2.90 (6H, m), 3.60 (2H, s), 4.01 (3H, s), 6.90 (1H,s), 7.07 (1H, d, J=8Hz), 7.32 (1H, dd, J=8, 1 Hz), 7.49 (1H, d, J=1Hz), 8.10 (1H, s), 9.80 (1H, br.s); ^{m}/z (API⁺): 337 (MH⁺).

### EXAMPLE 7.

N-(2-Methyl-1,2,3,4-tetrahydroisoquinolin-5-yl)-5-methoxy-1-indanone-6-carboxamide, hydrochloride

Prepared as in Example 5, but starting from 5-methoxy-indan-1-one-6-carboxylic acid P8.

¹H NMR (CDCl₃) [free base] δ: 2.48 (3H, s), 2.72 (6H, m), 3.13 (2H, m), 3.60 (2H, s), 4.13 (3H, s), 6.87 (1H, d, J=8Hz), 7.07 (1H, s), 7.20 (1H, t, J=8Hz), 8.11 (1H, d, J=8Hz), 8.70 (1H, s), 9.45 (1H, br.); ^{m}/z (API⁺): 351 (MH⁺).

### EXAMPLE 8.

N-(2-Methyl-1,2,3,4-tetrahydroisoquinolin-5-yl)-5-iso-butyroyl-2-methoxy-4-iso-propoxybenzamide, hydrochloride

¹H NMR (CDCl₃) [free base] δ: 1.15 (6H,d, J=7Hz), 1.51 (6H, d, J=7Hz), 2.47 (3H, s), 2.74 (2H, m), 2.83 (2H, m), 3.45 (1H, m), 3.61 (2H, m), 3.94 (3H, s), 4.90 (1H, m), 6.54 (1H, s), 6.87 (1H, s), 7.19 (1H, t, J=8Hz), 7.89 (1H, d, J=8Hz), 8.58 (1H, s), 9.28 (1H, br.s); ^{m}/z (API⁺): 425 (MH⁺; 100%).

### EXAMPLE 9.

N-(2-Methyl-1,2,3,4-tetrahydroisoquinolin-5-yl)-5-acetyl-2-methoxy-4-iso-propylbenzamide, hydrochloride

¹H NMR (CDCl₃) [free base] δ: 1.27 (6H,d, J=7Hz), 2.49 (3H, s), 2.64 (3H, s), 2.80 (4H, m), 3.61 (2H, s), 3.85 (1H, m), 4.13 (3H, s), 6.87 (1H, d, J=8Hz), 7.05 (1H, s), 7.21 (1H, t, J=8Hz), 8.13 (1H, d, J=8Hz), 8.67 (1H,s), 9.64 (1H, br.s); ^{m}/z (API⁺): 381 (MH⁺).

### EXAMPLE 10.

N-(2-Methyl-1,2,3,4-tetrahydroisoquinolin-5-yl)-5-cyano-2-methoxy-4-iso-propylbenzamide, hydrochloride

The free base was prepared in 50% yield from the amine of Description 7 using a procedure similar to that of Example 4.

¹H NMR (250MHz, CDCl₃) δ: 1.41 (6H,d, J=7Hz), 2.83 (4H, br.s), 3.48 (1H, sept, J=7Hz), 3.67 (2H, s), 4.19 (3H, s), 6.93 (1H,d, J=7.5), 7.26 (1H, dd, J=7.5, 7.5 Hz), 8.11 (1H,d, J=7.5Hz), 9.52 (1H, br.); ^{m}/z (API⁺): 364 (MH⁺; 100%).

The following Examples 11-15 are prepared using methods similar to those described in the above Descriptions, Preparations and Examples.

### EXAMPLE 11.

N-(2-Methyl-1,2,3,4-tetrahydroisoquinolin-5-yl)-2-ethoxy-4-iso-propyl-5-trifluoromethylbenzamide

¹H NMR (CDCl₃) 67 : 1.29 (6H,d, J=7Hz), 1.59 (3H, t, J=7Hz), 2.48 (3H, s), 2.75 (2H, m), 2.83 (2H, m), 3.40 (1H, m), 3.61 (2H, s), 4.38 (2H, q, J=7Hz), 6.88 (1H, d, J=8Hz), 7.06 (1H, s), 7.20 (1H, t, J=8Hz), 7.98 (1H, d, J=8Hz), 8.59 (1H, s), 9.44 (1H, br.); ^{m}/z (API⁺): 421 (MH⁺).

### EXAMPLE 12.

N-(2-Methyl-1,2,3,4-tetrahydroisoquinolin-5-yl)-2-n-propoxy-4-iso-propyl-⁵-trifluoromethylbenzamide, hydrochloride

¹H NMR (CDCl₃) [free base] δ: 1.09 (3H, t, J=7Hz), 1.28 (6H, d, J=7Hz),1.98 (2H, m), 2.48 (3H, s), 2.74 (2H, m), 2.81 (2H, m), 3.40 (1H, m), 3.61 (2H, s), 4.26 (2H, t, J=7Hz), 6.90 (1H, d, J=8Hz), 7.06 (1H, s), 7.20 (1H, t, J=8Hz), 7.90 (2H, d, J=8Hz), 8.58 (1H, s), 9.41 (1H, br.s); ^{m}/z (API⁺): 435 (MH⁺; 80%).

### EXAMPLE 13.

N-(2-Methyl-1,2,3,4-tetrahydroisoquinolin-5-yl)-2-iso-propoxy-4-iso-propyl-5-trifluoromethylbenzamide, hydrochloride

¹H NMR (CDCl₃) [free base] δ: 1.29 (6H, d, J=7Hz). 1.51 (6H, d, J=7Hz), 2.48 (3H, s), 2.74 (2H, m), 2.83 (2H, m), 3.41 (1H, m), 3.61 (1H,s), 4.93 (1H,m), 6.89 (1H, d, J=8Hz), 7.07 (1H, s), 7.20 (1H, t, J=8Hz), 7.90 (1H, d, J=8Hz), 8.58 (1H, s), 9.41 (1H, br.s); ^{m}/z (API⁺): 435 (MH⁺).

### EXAMPLE 14.

N-(2-Methyl-1,2,3,4-tetrahydroisoquinolin-5-yl)-2-n-butoxy-4iso-propyl-5-trifluoromethylbenzamide, hydrochloride

¹H NMR (CDCl₃) [free base] δ: 1.00 (3H, t, J=7Hz), 1.30 (6H, d, J=7Hz), 1.51 (2H, m), 1.91 (2H, m), 2.48 (3H, s), 2.75 (2H, m), 2.82 (2H, m), 3.41 (1H, m), 3.62 (2H, s), 4.30 (2H, t, J=7Hz), 6.89 (1H, d, J=8Hz), 7.06 (1H, s), 7.20 (1H, t, J=8Hz), 7.91 (1H,d, J=8Hz), 8.58 (1H, s), 9.41 (1H, br.s);

^{m}/z (API⁺): 449 (MH⁺).

### EXAMPLE 15.

N-(2-Methyl-1,2,3,4-tetrahydroisoquinolin-5-yl)-2-cyclopropylmethoxy-4-iso-propyl-5-trifluoromethylbenzamide, hydrochloride

Prepared by coupling 5-amino-2-methyl-1,2,3,4-tetrahydroisoquinoline D7 with 2-cyclopropylmethoxy-4-iso-propyl-5-trifluoromethyl benzoic acid.

¹H NMR (CDCl₃) [free base] 67 : 0.48 (2H, m), 0.75 (2H, m), 1.29 (6H, d, J=7Hz), 1.31 (1H, m), 2.45 (3H, s), 2.71 (2H, m), 2.89 (2H, m), 3.40 (1H, m), 3.61 (2H, s), 4.09 (2H, d, J=7Hz), 6.89 (2H, d, J=8Hz), 7.01 (1H, s), 7.21 (1H, t, J=8Hz), 7.97 (1H, d, J=8Hz), 8.61 (1H, s), 9.61 (1H, br.s); ^{m}/z (API⁺): 447 (MH⁺).

### EXAMPLE 16.

N-(2-Methyl-1,2,3,4-tetrahydroisoquinolin-5-yl)-5-cyano-2-ethoxy-4-iso-propylbenzamide, hydrochloride

The acid of Preparation 6b was converted into the acid chloride using a procedure similar to that described in Fieser and Fieser, Reagents for Organic Synthesis, vol 1, p.286 and coupled with the amine D7 using a procedure similar to that of Example 4 to give the title compound in 60% yield.

¹H NMR (250MHz, d⁶DMSO) δ: 1.41 (6H,d, J=7Hz), 1.24 (3H, t, J=7Hz), 6.91 (1H, d, J=7Hz), 7.06 (1H, s), 7.14 (1H, dd, J=7.5, 7.5Hz), 7.41 (1H, d, J=7.5Hz), 7.85 (1H, s), 9.60 (1H, s), 10.69 (1H, br); ^{m}/z (API⁺): 378 (MH⁺,38%), 755 (2MH+,100%).

The following Examples 17-20 are prepared using methods similar to those described in the above Descriptions, Preparations and Examples.

### EXAMPLE 17.

N-(2-Methyl-1,2,3,4-tetrahydroisoquinolin-5-yl)-5-bromo-4-ethoxy-2-methoxybenzamide

¹H NMR (CDCl₃) δ: 1.52 (3H, t, J=7Hz), 2.47 (3H, s), 2.78 (4H, m), 3.58 (2H, s), 4.05 (3H, s), 4.16 (2H, q, J=7Hz), 6.50 (1H, s), 6.84 (1H, d, J=8Hz), 7.20 (1H, t, J=8Hz), 8.11 (1H, d, J=8Hz), 8.48 (1H,s), 9.52 (1H, br.s);

^{m}/z (API⁺): 420 (MH⁺).

### EXAMPLE 18.

N-(2-Methyl-1,2,3,4-tetrahydroisoquinolin-5-yl)-2-methoxy4-iso-propyl-5-pentafluoroethylbenzamide, hydrochloride

¹H NMR (250MHz, d⁶DMSO) δ: 1.32 (6H,d, J=7Hz), 7.09 (1H, d, J=7.5Hz), 7.33 (1H, dd, J=7.5, 7.5Hz), 7.39 (1H, s), 7.76 (1H, d, J=7.5Hz), 7.97 (1H, s), 9.82 (1H, s), 10.80 (1H, br. s); ^{m}/z (API⁺): 457 (MH⁺,100%),

### EXAMPLE 19.

N-(2-Methyl-1,2,3,4-tetrahydroisoquinolin-5-yl)4-ethoxy-2-methoxy-5-trifluoromethylbenzamide

¹H NMR (CDCl₃) δ: 1.50 (3H, t, J=7Hz), 2.48 (3H, s), 2.77 (4H, m), 3.61 (2H, s), 4.10 (3H, s), 4.21 (2H, q, J=7Hz), 6.56 (1H, s), 6.85 (1H, d, J=8Hz), 7.20 (1H, t, J=8Hz), 8.11 (1H, d, J=8Hz), 8.57 (1H, s), 9.64 (1H, br.s);

^{m}/z (API⁺): 409 (MH⁺; 100%).

### EXAMPLE 20.

N-(2-Methyl-1,2,3,4-tetrahydroisoquinolin-7-yl)-2,4-dimethoxy-5-trifluoromethylbenzamide

¹H NMR (250MHz, CDCl₃) [free base]δ: 2.46 (3H, s), 2.69 (2H, t, J=6Hz), 2.90 (2H, t, J=6Hz), 3.60 (2H, s), 3.99 (3H, s), 4.12 (3H, s), 6.57 (1H,s), 7.09 (1H, d, J=8Hz), 7.27 (1H, m), 7.46 (1H, d, J=2Hz). 8.54 (1H ,s), 9.43 (1H,brs);

^{m}/z (API⁺): 395 (MH⁺; 100%).

### EXAMPLE 21.

N-(2-Methyl-1,2,3,4-tetrahydroisoquinolin-5-yl)-4-n -butoxy-2-methoxy-5-cyanobenzamide, hydrochloride.

¹H NMR (free base 250 MHz, CDCl₃) δ: 1.01 (3H, t, J=7.5 Hz), 1.50-1.65 (2H, m), 1.80-1.85 (2H, m), 2.47 (3H, s), 2.77 (4H, brs), 3.60 (2H, s), 4.10-4.15 (5H, m), 6.51 (1H, s), 6.86 (1H, d, J=8 Hz), 7.20 (1H, dd, J=8, 8 Hz), 8.06 (1H, d, J=8Hz), 8.55 (1H, s), 9.34 (1H, s); ^{m}/z (API⁺): 394 (MH⁺,394).

### EXAMPLE 22.

N-(2-Methyl-1,2,3,4-tetrahydroisoquinolin-S—yl)-4-ethoxy-2-methoxy-S—cyanobenzamide, hydrochloride.

¹H NMR (free base 250MHz, d⁶DMSO) δ: 1.40 (3H, t, J 7 Hz), 2.32 (3H, s), 2.55-2.65 (2H, m), 2.70-2.75 (2H, m), 3.47 (2H, s), 4.33 (2H, q, J=7 Hz), 6.87 (1H, d, J=7.5 Hz), 6.92 (1H, s), 7.13 (1H, dd, J=7.5, 7.5 Hz), 7.69 (H, d, J=7.5 Hz), 8.12 (1H, s), 9.56 (1H, s); ^{m}/z (API⁺): 366 (MH⁺,100%)

### EXAMPLE 23.

N-(2-Methyl-1,2,3,4-tetrahydroisoquinolin-5-yl)-2,4-dimethoxy-5-cyanobenzamide, hydrochloride.

¹H NMR (free base 250MHz, CDCl₃) δ2.49 (3H, s), 2.80 (4H, s), 3.62 (2H, s), 4.02 (3H, s), 4.14 (3H, s), 6.53 (1H, s), 6.88 (1H, d, J=7 Hz), 7.21 (1H, t, J=7 Hz), 8.06 (1H, d, J=7Hz), 8.56 (1H, s), 9.30 (1H, br); ^{m}/z (API⁺): 352 (MH⁺,100%).

### EXAMPLE 24.

N-(2-Methyl-1,2,3,4-tetrahydroisoquinolin-5-yl)-2-methoxy-4-iso-propoxy-S—cyanobenzamide, hydrochloride.

¹H NMR (free base 250MHz, CDCl₃) δ: 1.46 (6H, d, J=6 Hz), 2.47 (2H, s), 2.77 (3H, br), 3.60 (2H, s), 4.10 (3H, s), 4.74 (1H, sept, J=6 Hz), 6.52 (1H, s), 6.86 (1H, d, J=7.5 Hz), 7.20 (1H, dd, J=7.5, 7.5 Hz), 8.06 (1H, d, J=ca 8 Hz), 8.55 (1H, s), 9.34 (1H, br s);^{m}/z (API⁺): 380 (MH⁺,100%c).

### EXAMPLE 25.

N-(2-Methyl-1,2,3,4-tetrahydroisoquinolin-5-yl)-2,4-diethoxy-5-bromobenzamide hydrochloride.

¹H NMR (250MHz, CDCl₃) [free base]δ: 1.52 (3H, t, J=7Hz), 1.56 (3H, t, J=7Hz), 2.47 (3H, s), 2.74 (2H, m), 2.82 (2H, m), 3.61 (2H, s), 4.15 (2H, sq, J=7Hz), 4.29 (2H, q, J=7Hz), 6.51 (1H, s), 6.87 (1H, d, J=8Hz), 7.19 (1H, t, J=8Hz), 7.96 (1H, d, J=8Hz), 8.49 (1H,s), 9.40 (1H,brs); ^{m}/z (API⁺): 435, 433 (MH⁺; 80%).

### EXAMPLE 26.

N-(2-Methyl-1,2,3,4-tetrahydroisoquinolin-5-yl)-5-acetyl-2,4-diethoxybenzamide, hydrochloride.

¹H NMR (free base CDCl₃) δ:1.55 (6H, m), 2.49 (3H, s), 2.60 (3H, s), 2.78 (4H, m), 3.63 (2H, s), 4.18 (2H, q, J=7 Hz), 4.33 (2H, q, J=7 Hz), 6.49 (1H, s), 6.86 (1H, d, J=7 Hz), 7.19 (1H, t, J=7 Hz), 7.97 (1H, d, J=7 Hz), 8.76 (1H, s), 9.22 (1H, br. s).

^{m}/z (API⁺) 397 (MH⁺,100%).

### EXAMPLE 27

N-(2-Methyl-1,2,3,4-tetrahydroisoquinolin-5-yl )-4-ethoxy-2-methoxy-5-methylsulfonylbenzamide

¹H NMR (CDCl₃) δ: 1.56 (3H, t), 2.47 (3H, s), 2.88 (4H, s), 3.18 (3H, s), 3.59 (2H, s), 4.11 (3H, s), 4.28 (2H, q), 6.55 (1H, s), 6.83 (1H, d), 7.17 (1H, t), 8.09 (1H, d), 8.88 (1H, s), 9.29 (1H, s)

### EXAMPLE 28

N-(2-Methyl-1,2,3,4-tetrahydroisoquinolin-5-yl)-4-iso -propyl-2-methoxy-5-methysulfonylbenzamide

¹H NMR (CDCl₃) δ: 1.37 (6H, d), 2.48 (3H, s), 2.77 (4H, m), 3.12 (3H, s), 3.61 (2H, s), 4.00(1H, m), 4.15(3H, s), 6.87(1H, d), 7.11 (1H, s), 7.21 (1H, t), 8.10(1H, d), 8.94(1H, s), 9.42 (1H, s); ^{m}/z (API⁺) : 417.1 (MH⁺; 92%).

### EXAMPLE 29

N-(2-Methyl-1,2,3,4-tetrahydroisoquinolin-5-yl)-2,4-dimethoxy-5-methylsulfonylbenzamide

¹H NMR (CDCl₃) δ: 2.48 (3H, s), 2.78 (4H, s), 3.18 (3H, s), 3.59 (2H, s), 4.08 (3H, s), 4.14 (3H, s), 6.59 (1H,s), 6.85 (1H, d), 7.19 (1H, t), 8.10 (1H, d), 8.91 (1H, s), 9.29 (1H, s); ^{m}/z (API⁺): 405.1 (MH⁺; 100%)

### EXAMPLE 30

N-(2-Methyl-1,2,3,4-tetrahydroisoquinolin-5-yl)-2-methoxy-4-methyl-5-methylsulfonylbenzamide

¹H NMR (CDCl₃) δ: 2.48 (3H, s), 2.78 (7H, m), 3.09 (3H, s), 3.60 (2H, s), 4.13 (3H, s), 6.88 (1H, d), 6.96 (1H, s), 7.21 (1H, t), 8.10 (1H, d), 8.30 (1H, s), 9.41 (1H, s).

^{m}/z (API⁺): 389.1 (MH⁺; 100%).

### EXAMPLE 31

N-(2-Methyl-1,2,3,4-tetrahydroisoquinolin-5-yl)-2-ethoxy4-ethyl-5-methylsulfonylbenzamide

¹H NMR (CDCl₃) δ: 1.37 (3H, t), 1.61 (3H, t), 2.48 (3H, s), 2.75 (2H, t), 2.81 (2H, t), 3.11 (5H, m), 3.61 (2H, s), 4.40 (2H, q), 6.89 (1H, d), 7.00 (1H, s), 7.21 (1H,t), 7.97 (1H, d), 8.94 (1H, s), 9.29 (1H, s); ^{m}/z (API⁺): 417.1 (MH⁺, 100%).

### EXAMPLE 32

N-(2-Methyl-1,2,3,4-tetrahydroisoquinolin-5-yl)-5-cyano-2-ethoxy-4-methylbenzamide

¹H NMR (CDCl₃) δ: 1.55 (3H, t), 2.44 (3H, s), 2.47 (3H, s), 2.73 (2H, t), 2.83 (2H, t), 3.61 (2H, s), 4.28 (2H, q), 6.84 (1H, d), 6.90 (1H, s), 7.20 (1H, t), 7.98 (1H, d), 8.45 (1H, s), 9.49 (1H, s); ^{m}/z (API⁺): 350.1 (MH⁺; 100%).

### EXAMPLE 33

N-(2-Methyl-1,2,3,4-tetrahydroisoquinolin-5-yl)-5-cyano-2-methoxy-4-methylbenzamide

¹H NMR (CDCl₃) δ: 2.48 (3H, s), 2.62 (3H, s), 2.78 (4H, m), 3.60 (2H, s), 4.12 (3H, s), 6.88 (1H, d), 6.96 (1H, s), 7.21 (1H, t), 8.08 (1H, d), 8.58 (1H, s), 9.46 (1H,s).

^{m}/z (API⁺): 336.1 (MH⁺; 100%).

### EXAMPLE 34

N-(2-Methyl-1,2,3,4-tetrahydroisoquinolin-5-yl)-2,4-diethoxy-5-cyanobenzamide hydrochloride

¹H NMR (free base 250 MHz, CDCl₃) δ1.52 (3H, t, J=7 Hz), 1.60 (3H obscured by H₂O, t), 2.47 (3H, s), 2.75 (4H, m), 3.60 (2H, s), 4.20 (2H, q, J=7 Hz), 4.35 (2H, q, J=7 Hz), 6.50 (1H, s), 6.89 (1H, d, J=7.5 Hz), 7.20 (1H, dd, J=7.5, 7.5 Hz), 7.92 (1H, d, J=7.5 Hz), 8.56 (1H, s), 9.21 (1H, br); ^{m}/z (API⁺): 380 (MH⁺, 100%)

### EXAMPLE 35

N-(2-Methyl-1,2,3,4-tetrahydroisoquinolin-5-yl)-2-ethoxy-4-ethyl-5-cyanobenzamide, hydrochloride

¹H NMR (250 MHz, d⁶DMSO) δ: 1.40 (3H, t, J=7.5 Hz), 1.55 (3H, t, J=7 Hz), 2.90-3.10 (5H, m), 4.40-4.55 (3H, m), 4.65 (1H, brd, J=15Hz), 7.22 (1H, d,=7.5Hz), 7.40-7.50 (2H, m), 7.72 (1H, d, J=8 Hz), 8.17 (1H, s), 9.90 (1H, s), 11.00 (1H, br s).

^{m}/z (API⁺): 364 (MH⁺, 100%).

### EXAMPLE 36

N-(2-Methyl-1,2,3,4-tetrahydroisoquinolin-5-yl)-2-methoxy-4-ethyl-5-cyanobenzamide, hydrochloride

¹H NMR (250 MHz, d⁶DMSO) δ: 1.25 (3H, t, J=7.5Hz), 2.80 (5H, m), 3.65 (1H, br), 4.01 (3H, s), 4.29 (1H, dd, J=15.5, 7.5 MHz), 4.49 (1H, d, J=ca 15Hz), 7.05 (1H, d, J=8 Hz), 7.28 (2H, m), 7.63 (1H, d, J=8 Hz), 8.05 (1H,s), 9.80 (1H,s), 10.9 (1H, br).

^{m}/z (API⁺): 350 (MH⁺, 100%).

### EXAMPLE 37

N-(2-Methyl-1,2,3,4-tetrahydroisoquinolin-5-yl)-2-ethoxy-4-ethyl-5-bromobenzamide, hydrochloride

¹H NMR (250 MHz, d⁶DMSO) δ: 1.10 (3H, t, J=7.5 Hz), 1.33 (3H, t, J=7 Hz), 2.65 (2H, q, J=7.5 Hz), 2.81 (3H, s), 2.96 (2H, br), 4.18 (2H, q, J=7 Hz), 4.30 (2H, br), 6.97 (1H, d, J=8 Hz), 7.13 (1H, s), 7.23 (1H, t, J=8 Hz), 7.60 (1H, d, J=8Hz), 7.81 (1H, s), 9.64 (1H, s); ^{m}/z (API⁻): 419/417 (MH⁻, 95/100%).

### EXAMPLE 38

N-(2-Methyl-1,2,3,4-tetrahydroisoquinolin-5-yl)-5-acetyl-2-ethoxy-4-ethylbenzamide, hydrochloride

¹H NMR (250 MHz, d⁶DMSO) δ: 0.97 (3H, t, J=7.5 Hz), 1.25 (3H, t, J=7 Hz), 2.38 (3H, s), 4.05-4.20 (3H, m), 4.32 (1H, d, J=15 Hz), 6.88 (1H, d, J=7.5 Hz), 6.95 (1H, s), 7.13 (1H, t, J=7.5, ca 7.5Hz), 7.54 (1H, d, J=8 Hz), 8.08 (1H, s), 9.53 (1H, s), 10.77 (1H, br);

^{m}/z (API⁺): 381 (MH⁺, 100%).

### EXAMPLE 39

N-(2-Methyl-1,2,3,4-tetrahydroisoquinolin-S—yl)-2-ethoxy4-ethyl-5-trifluoromethylbenzamide, hydrochloride

¹H NMR (250 MHz, d⁶DMSO) 67 : 1.05 (3H, t, J=7.5 Hz), 1.25 (3H, t, J=7 Hz), 2.50-2.75 (5H, m), 3.50 (1H, br), 4.10 (3H, m), 4.30 (1H, brd, J=15 Hz), 6.88 (1H, d, J=8 Hz), 7.12(2H, m), 7.49 (1H, d, J=8 Hz), 7.82 (1H, s), 9.55 (1H, s), 10.7 (1H, br).

^{m}/z (API⁺): 407 (MH⁺, 100%)

### EXAMPLE 40

N-(2-Methyl-1,2,3,4-tetrahydroisoquinolin-5-yl)-2-ethoxy-4-ethyl-5-fluorobenzamide

¹H NMR (250 MHz, CDCl₃) 67 : 1,25 (3H, t, J=7.5 Hz), 1.53 (3H, t, J=ca 7 Hz), 2.47 (3H, s), 2.76 (6H, m), 3.61 (2H, s), 4.28 (2H, q, J=7 Hz), 6.85 (2H, m), 7.20 (1H,t, J=ca 7.5 Hz), 7.96 (1H, d, J=10 Hz), 7.98 (1H, d, J=ca 7.5 Hz), 9.62 (1H, br s).

^{m}/z (API⁺): 357 (MH⁺, 100%)

### EXAMPLE 41

N-(2-Methyl-1,2,3,4-tetrahydroisoquinolin-5-yl)-5-bromo-2-ethoxy-4-methylbenzamide

¹H NMR (CDCl₃) δ: 1.59 (3H, t), 2.48 (3H, s), 2.60 (3H, s), 2.74 (2H, t), 2.82 (2H, t), 3.61 (2H, s), 4.37 (2H, q), 6.90 (1H, d), 6.95 (1H,s), 7.21 (1H, t), 7.94 (1H, d), 8.59 (1H, s), 9.33 (1H, s); ^{m}/z (API⁺): 403.0 (M⁺, 100%)

### EXAMPLE 42

N-(2-Methyl-1,2,3,4-tetrahydroisoquinolin-5-yl)-5-chloro-2-ethoxy-4-ethylbenzamide

¹H NMR (CDCl₃) δ: 1.26 (3H, t), 1.55 (3H, t), 2.47 (3H, s), 2.78 (6H, m), 3.60 (2H, s), 4.30 (2H, q), 6.87 (2H, d), 7.20 (1H, t), 7.98 (1H. d), 8.28 (1H, s), 9.52 (1H, s).

^{m}/z (APT⁺): 373.1 (MH⁺: 100%).

### EXAMPLE 43

N-(2-Methyl-1,2,3,4-tetrahydroisoquinolin-5-yl)-5-chloro-2-ethoxy-4-methylbenzamide

¹H NMR (CDCl₃) δ: 1.54 (3H, t), 2.42 (3H, s), 2.47 (3H, s), 2.74 (2H, t), 2.84 (2H, t), 3.61 (2H, s), 4.28 (2H, q), 6.87 (2H, d), 7.20 (1H, t), 7.98 (1H, d), 8.28 (1H, s), 9.51 (1H, s).

^{m}/z (API⁺): 359.1 (MH⁺; 100%).

### EXAMPLE 44

N-(2-Methyl-1,2,3,4-tetrahydroisoquinolin-5-yl)-2-ethoxy-4-methyl-5-trifluoromethylbenzamide

¹H NMR (CDCl₃) δ: 1.58 (3H, t), 2.48 (3H, s), 2.53 (3H, s), 2.74 (2H, t), 2.84 (2H, t), 3.61 (2H, s), 4.36 (2H, q), 6.88 (1H, d), 6.92 (1H, s), 7.21 (1H, t), 7.98 (1H, d), 8.60 (1H, s), 9.44 (1H, s); ^{m}/z (API⁺): 393.1 (MH⁺; 100%).

### EXAMPLE 45

N-(2-Methyl-1,2,3,4-tetrahydroisoquinolin-5-yl)-5-chloro-2-ethoxy-4-iso-propylbenzamide

¹H NMR (CDCl₃) δ: 1.27 (6H, d), 1.56 (3H, t), 2.47 (3H, s), 2.74 (2H, t), 2.84 (2H, t), 3.43 (1H, m), 3.61 (2H, s), 4.32 (2H, q), 6.88 (1H, d), 6.93 (1H, s), 7.20 (1H, t), 7.98 (1H, d), 8.28 (1H, s), 9.52 (1H, s); ^{m}/z (API⁺): 387.2 (MH⁺; 100%).

### EXAMPLE 46

N-(2-Methyl-1,2,3,4-tetrahydroisoquinolin-5-yl)-2-methoxy-4-iso-propoxy-5-trifluoroacetylbenzamide hydrochloride

¹H NMR (free base CDCl₃) δ:1.46 (6H, d, J=6 Hz), 2.48 (3H, s), 2.75 (4H, m), 3.60 (2H, s), 4.12 (3H, s), 4.79 (1H, m), 6.53 (1H, s), 6.85 (1H, d, J=6Hz), 7.16 (1H, t, 3=6 Hz), 8.08 (1H, d, J=6 Hz), 9.30 (1H, br. s); ^{m}/z (API⁺): 451 (MH⁺; 100%).

### EXAMPLE 47

N-(2-Methyl-1,2,3,4-tetrahydroisoquinolin-5-yl)-2,4-diethoxy-5-trifluoromethylbenzamide hydrochloride

¹H NMR (free base CDCl₃) δ: 1.52 (3H, t, J=7 Hz), 1.59 (3H, t, J=7 Hz), 2.47 (3H, s), 2.74 (2H, m), 2.80 (2H, m), 3.60 (2H, s), 4.17 (2H, q, J=7 Hz), 4.33 (2H, q, J=7 Hz), 6.55 (1H, s), 6.87 (1H, d, J=7 Hz), 7.20 (1H, d, J=7 Hz), 7.96 (1H, d, J=7 Hz), 8.57 (1H, s), 9.32 (1H, br. s); ^{m}/z (API⁺) 423 (MH⁺; 100%).

### EXAMPLE 48

N-(2-Methyl-1,2,3,4-tetrahydroisoquinolin-5-yl)-2,4-diethoxy-5-trifluoroacetyl-benzamide hydrochloride

¹H NMR (DMSO-d₆) δ: 1.37 (3H, t, J=7 Hz), 1.45 (3H, d, J=7 Hz), 2.90 (3H, s), 3.03 (4H, brs), 3.76 (2H, brs), 4.30 (2H, q, J=7 Hz), 4.42 (2H, q, J=7 Hz), 6.88 (1H, s), 7.05 (1H, d, 3=7 Hz), 7.30 (1H, t, J=7 Hz), 7.64 (1H, d, J=7 Hz), 8.18 (1H, s), 9.60 (1H, s), 10.55 (1H, brs); ^{m}/z (API⁺) 451 (MH⁺; 100%).

### EXAMPLE 49

N-(2-Methyl-1,2,3,4-tetrahydroisoquinolin-5-yl)-5-cyano-4-ethyl-2-n-propoxybenzamide

¹H NMR (CDCl₃) δ: 1.09 (3H, t, J=8 Hz), 1.34 (3H, t, J=8 Hz), 1.97 (2H, m), 2.48 (3H, s), 2.74 (2H, t, J=7 Hz), 2.81 (2H, t, J=7 Hz), 3.62 (2H, s), 4.26 (2H, t, J=8 Hz), 6.91 (1H, d), 6.96 (1H, s), 7.21 (1H, t), 7.86 (1H, d), 8.59 (1H, s), 9.31 (1H, s); ^{m}/z (API⁺): 378.2 (MH⁺; 100%).

### EXAMPLE 50

N-(2-Methyl-1,2,3,4-tetrahydroisoquinolin-5-yl)-2-n-butoxy-5-cyano-4-ethylbenzamide

¹H NMR (CDCl₃) δ: 1.00 (3H, t, J=8 Hz), 1.34 (3H, t, l=8 Hz), 1.51 (2H, m), 1.93 (2H, m), 2.48 (3H, s), 2.78 (4H, m), 2.92 (2H, q, J=8 Hz), 3.62 (2H, s), 4.31 (2H, t, 1=8 Hz), 6.91 (1H, d), 6.96 (1H, s), 7.21 (1H, t), 7.86 (1H, d), 8.58 (1H, s), 9.30 (1H, s); ^{m}/z (API⁺): 392.2 (MH⁺; 100%).

### EXAMPLE 51

N-(2-Methyl-1,2,3,4-tetrahydroisoquinolin-7-yl)-2-n-butoxy-5-cyano-4-ethylbenzamide

¹H NMR (CDCl₃) δ: 1.06 (3H, t, J=8 Hz), 1.34 (3H, t, J=8 Hz), 1.64 (2H, m), 2.01 (2H, m), 2.47 (3H, s), 2.69 (2H, t, J=7 Hz), 2.91 (4H, m), 3.60 (2H, s), 4.28 (2H, t, J=8 Hz), 6.9 (1H, s), 7.10 (1H, d), 7.25 (1H, dd, J=7, 2 Hz), 7.45 (1H, d), 8.55 (1H, s), 9.70 (1H, s); ^{m}/z (API⁺): 392.2 (MH⁺; 80%).

### EXAMPLE 52

N-(2-Methyl-1,2,3,4-tetrahydroisoquinolin-S—yl)-2-ethoxy-5-cyano-4-iso-propoxybenzamide

¹H NMR (DMSO-d₆) 67 : 1.19-1.25 (9H, m), 2.71 (3H, s), 2.88 (2H, br), 4.18 (4H, brm), 4.91 (1H, sep, J=6 Hz), 6.74 (1H, s), 6.90 (1H, d, J=8 Hz), 7.13 (1H, dd, J=8, 8 Hz), 7.42 (1H, d, J=8 Hz), 7.87 (1H, s), 9.39 (1H, s), 10.70 (1H, br);

^{m}/z (API⁺): 394 (MH⁺; 100%).

### EXAMPLE 53

N-(2-Methyl-1,2,3,4-tetrahydroisoquinolin-5-yl)-5-dimethylaminocarbonyl-4-ethyl-2-methoxybenzamide

¹H NMR (CDCl₃) δ: 1.26 (3H, t, J=8 Hz), 2.48 (3H, s), 2.74 (6H, m), 2.88 (2H, s), 3.13 (3H, s), 3.60 (2H, s), 4.08 (3H, s), 6.85 (1H, d, J=8 Hz), 6.92 (1H, s), 7.19 (1H, t, J=8 Hz), 8.13 (2H, m), 9.67 (1H, brs); ^{m}/z (API⁺): 396 (MH⁺; 100%).

### EXAMPLE 54

N-(2-Methyl-1,2,3,4-tetrahydroisoquinolin-5-yl)-2,4-diethoxy-5-pivaloylbenzamide

¹H NMR (CDCl₃) δ: 1.23 (9H, s), 1.42 (3H, t, J=7 Hz), 1.57 (3H, t, J=7 Hz), 2.47 (3H, s), 2.74 (2H, m), 2.82 (2H, m), 3.60 (2H, s), 4.08 (2H, q, J=7 Hz), 4.31 (2H, q, J=7 Hz), 6.49 (1H, s), 6.86 (1H, d, J=7 Hz), 7.19 (1H, t, J=7 Hz), 7.99 (1H, t, J=7 Hz), 8.07 (1H, s), 9.43 (1H, br.s); ^{m}/z (API⁺) 439 (MH⁺; 80%).

### EXAMPLE 55

N-(2-Methyl-1,2,3,4-tetrahydroisoquinolin-5-yl)-2,4-diethoxy-5-iso-butyroylbenzamide

¹H NMR (DMSO-d₆) δ: 0.93 (6H, d, J=7 Hz), 1.29 (3H, t,=7 Hz), 1.34 (3H, t, J=7 Hz), 2.78-3.60 (8H, m), 4.20 (6H, m), 6.69 (1H, s), 6.91 (1H, d, J=7 Hz), 7.49 (1H, t, J=7 Hz), 7.62 (1H, d, l=7 Hz), 7.99 (1H, s), 9.45 (1H, s), 10.67 (1H, brs); ^{m}/z (API⁺) 425 (MH⁺; 90%).

### EXAMPLE 56

N-(2-Methyl-1,2,3,4-tetrahydroisoquinolin-S—yl)-2-methoxy-4-iso-propyl-5-trifluoromethylsulfonylbenzamide

¹H NMR (CDCl₃) δ: 1.34 (6H, d, J=7 Hz), 2.98-3.10 (4H, m), 3.23 (1H, br), 3.45 (1H, br), 3.69 (1H, br), 4.00 (2H, m), 4.21 (3H, s), 4.58 (1H, m), 7.00 (1H, d, J=8 Hz), 7.21 (1H, s), 7.33 (1H, dd, J=8, 8 Hz), 7.76 (1H, d, J=8 Hz), 8.97 (1H, s), 9.23 (1H, brs), 13.20 (1H, br); ^{m}/z (API⁺): 471 (MH⁺; 100%).

### EXAMPLE 57

N-(2-Hydroxyethyl-1,2,3,4-tetrahydroisoquinolin-5-yl)-5-bromo-4-ethoxy-2-methoxybenzamide

¹H NMR (CDCl₃) 67 : 1.54 (3H. t, J=7 Hz), 2.74 (2H, t, J=5 Hz), 2.82 (2H, d, J=5 Hz), 2.88 (2H, d, J=5 Hz), 3.73 (4H, m), 4.07 (3H, s), 4.18 (2H, q, J=7 Hz), 6.52 (1H,s), 6.87 (1H, d J=8 Hz), 7.21 (1H, t, J=8 Hz). 8.08 (1H, d.=8 Hz), 8.49 (1H, s), 9.51 (1H, s); ^{m}/z (API⁺): 449.451 (MH⁺; 80%).

### EXAMPLE 58

N-(2-Hydroxyethyl-1,2,3,4-tetrahydroisoquinolin-5-yl)-S—chloro-2-methoxy-4-iso-propoxybenzamide

¹H NMR (CDCl₃) δ: 1.44 (6H, d, J=6 Hz), 2.74 (2H, t, J=5 Hz), 2.82 (2H, d, J=5 Hz), 2.90 (2H, d, J=5 Hz), 3.70 (4H, m), 4.06 (3H, s), 4.62 (1H, m), 6.58 (1H, s), 6.87 (1H, d, J=8 Hz), 7.21 (1H, t, J=8 Hz), 8.09 (1H, d, J=8 Hz), 8.32 (1H, s), 9.52 (1H, brs); ^{m}/z (API⁺): 419, 421 (MH⁺).

### EXAMPLE 59

N-(2-Hydroxyethyl-1,2,3,4-tetrahydroisoquinolin-5-yl)-S—acetyl-2,4-diethoxybenzamide

¹H NMR (CDCl₃) δ: 1.52 (3H, t, J=7 Hz), 1.58 (3H, t, J=7 Hz), 1.97 (1H, brs), 2.59 (3H, s), 3.23 (1H, br), 2.73 (2H, t, J=5 Hz), 2.69-2.92 (4H, m), 3.75 (4H, m), 4.18 (2H, q, J=7 Hz), 4.34 (2H, q, J=7 Hz), 6.48 (1H, s), 6.87 (1H, d, J=8 Hz), 7.20 (1H, t, J=8 Hz), 7.96 (1H, d, J=8 Hz), 8.75 (1H, s), 9.20 (1H, brs);

^{m}/z (API⁺): 427 (MH⁺; 100%).

### EXAMPLE 60

N-(2-Methyl-l,3,4-tetrahydroisoquinolin-7-yl)-4-hydroxymethylbenzamide

¹H NMR (DMSO-d₆) δ: 2.35 (3H, s), 2.59 (2H, t), 2.78 (2H, t), 3.47 (2H, s), 4.58 (2H, d, J=7Hz), 5.33 (1H, t, J=7Hz), 7.06 (1H,d, J=8 Hz), 7.46 (3H, m), 7.92 (2H, d, J=8 Hz), 10.07 (1H,s); ^{m}/z (ES⁺) 297.1 (MH⁺; 70%).

### EXAMPLE 61

N-(1,2,3,4-Tetrahydroisoquinolin-7-yl)-3-aminobenzamide

¹H NMR (DMSO-d₆) δ: 2.71 (2H, m), 3.08 (2H, m), 3.98 (2H, m), 5.24 (2H, m), 6.64 (1H, m), 7.03 (4H, m), 7.43 (2H, m), 9.98 (1H, s);

^{m}/z (API⁺) 268 (MH⁺; 100%).

### EXAMPLE 62

N-(1,2,3,4-Tetrahydroisoquinolin-7-yl)-3-trifluoromethyldiazirinylbenzamide

¹H NMR (CDCl₃) δ: 2.78 (2H, t, J=7 Hz), 3.14 (2H, t, J=7 Hz), 4.01 (2H, s), 7.08 (1H, d, J=7 Hz), 7.20-7.60 (4H. m), 7.57 (1H, s), 7.76 (1H, s), 7.87 (1H, dd, J=1, 7 Hz); ^{m}/z (API⁺): 361 (MH⁺; 80%).

### EXAMPLE 63

N-(2-Methyl-2,3,4,5-tetrahydro-1H-benzo[c]azepin-8-yl)-4-methoxy-3-trifluoromethylbenzamide

¹H NMR (CDCl₃) δ: 1.81 (2H, m), 2.37 (3H, s), 2.89 (2H, m), 3.12 (2H, m), 3.92 (2H, s), 3.98 (3H, s), 7.00-7.15 (2H. m) 7.43-7.55 (2H, m), 8.02 (1H,s), 8.07-8.18(2H, m); ^{m}/z (API⁺): 379 (MH⁺: 100%).

### EXAMPLE 64

N-(2-Methyl-2,3,4,5-tetrahydro-1H-benzo[c]azepin-8-yl)-3-cyano-4-methoxy-benzamide

¹H NMR (CDCl₃) δ:1.78 (2H, m), 2.35 (3H, s), 2.88 (2H, m), 3.07 (2H, m), 3.86 (2H, s), 4.02 (3H, s), 7.07 (1H, d, J=8 Hz), 7.14 (1H, d, J=8 Hz), 7.38-7.47 (2H, m), 7.90 (1H, brs), 8.08-8.17 (2H, m); ^{m}/z (API⁺): 336 (MH⁺; 100%).

### EXAMPLE 65

N-(2-Methyl-2,3,4,5-tetrahydro-1H-benzo[c]azepin-8-yl)-3-chloro-4-ethoxy-benzamide

¹H NMR(CDCl₃) δ:1.49 (3H, t, J=7 Hz), 1.82 (2H, m), 2.36 (3H, s), 2.87 (2H, m), 3.15 (2H, m), 3.96 (2H, s), 4.15 (2H, q, J=7 Hz), 6.93 (1H, d, J=8 Hz), 7.10 (1H,d, J=8 Hz), 7.43 (1H, d, J=2 Hz), 7.58 (1H, dd, J=8,2 Hz), 7.80 (1H, dd, J=8, 2 Hz), 7.95 (1H, d, J=2 Hz), 8.34 (1H, br); ^{m}/z (API⁺): 359 (MH⁺;100%).

### EXAMPLE 66

N-(2-Methyl-2,3,4,5-tetrahydro-1H-benzo[c]azepin-8-yl)-3-bromo-4-ethoxy-benzamide

¹H NMR (CDCl₃) δ:1.51 (3H, t, J7 Hz), 1.77 (2H, m), 2.33 (3H, s), 2.87 (2H, m), 3.07 (2H, m), 3.86 (2H, s), 4.18 (2H, q, J=7 Hz), 6.94 (1H, d, J=8 Hz), 7.12 (1H, d,=8 Hz), 7.38-7.47 (2H, m), 7.71 (1H, s), 7.81 (1H, dd, J=8, 2 Hz), 8.05 (1H, d, J=2 Hz); ^{m}/z (API⁺): 405, 403 (MH⁺; 100%).

### EXAMPLE 67

N-(2-Methyl-2,3,4,5-tetrahydro-1H-benzo[c]azepin-8-yl)-3-acetyl-4-ethoxy-benzamide

¹H NMR (CDCl₃) δ: 1.51 (3H, t, J=7 Hz), 1.77 (2H, m), 2.33 (3H, s), 2.66 (3H, s), 2.87 (2H, m), 3.07 (2H, m), 3.87 (2H, s), 4.22 (2H, q, J=7 Hz), 7.03 (1H, d, J=8 Hz), 7.10 (1H, d, J=8 Hz), 7.42-7.52 (2H, m), 8.14 (1H, dd, J=8, 2 Hz), 8.21 (2H, m); ^{m}/z (API⁺): 367.2 (MH⁺; 100%).

### EXAMPLE 68

N-(2-Methyl-2,3,4,5-tetrahydro-1H-benzo[c]azepin-8-yl)-3-cyano-4-ethyl-benzamide

¹H NMR (CDCl₃) δ: 1.32 (3H, t, J=7 Hz), 1.80 (2H, m), 2.37 (3H, s), 2.88 (2H, m), 2.93 (2H, q, J=7 Hz), 3.11 (2H, m), 3.91 (2H, s), 7.12 (1H, d, J=8 Hz), 7.39-7.49 (2H, m), 7.54 (1H, dd, J=8, 2 Hz), 8.06 (1H, dd, J=8, 2 Hz); 8.17 (1H, d, J=2 Hz), 8.42 (1H, brs); ^{m}/z (API⁺): 334.2 (MH⁺; 100%).

### EXAMPLE 69

N-(2-Methyl-2,3,4,5-tetrahydro-1H-benzo[c]azepin-8-yl)-3-bromo-4-iso-propylbenzamide

¹H NMR (CDCl₃) 67 : 1.25 (6H, d, 3=7 Hz), 1.77 (2H, m), 2.31 (3H, s), 2.85 (2H, m), 3.06 (2H, m), 3.40 (1H, m), 3.84 (2H, s), 7.10 (1H, d, J=8 Hz), 7.35 (1H, d, J=8 Hz), 7.40-7.51 (2H, m), 7.79 (1H, dd, J=8, 2 Hz), 8.04 (1H, d, J=2 Hz); 8.08 (1H, brs); ^{m}/z (API⁺): 403, 401 (MH⁺; 100%).

### EXAMPLE 70

N-(2-Methyl-2,3,4,5-tetrahydro-1H-benzo[c]azepin-8-yl)-3-acetyl-4-iso-propylbenzamide

¹H NMR (CDCl₃) δ: 1.27 (6H, d, J=7 Hz), 1.79 (2H, m), 2.34 (3H, s), 2.63 (3H, s), 2.88 (2H, m), 3.09 (2H, m), 3.49 (2H, m), 3.88 (2H, s), 7.14 (1H, d, J=8 Hz), 7.39-7.60 (3H, m), 7.87 (1H, dd, J=8, 2 Hz), 7.90 (1H, brs), 8.02 (1H, d, J=2 Hz); ^{m}/z (API⁺): 365 (MH⁺; 100%).

### PHARMACOGICAL DATA

1. Binding Assay Method

WO 92/22293 (SmithKline Beecham) discloses compounds having anti-convulsant activity, including inter alia the compound trans-(+)-6-acetyl-4S-(4-fluorobenzoylamino)-3,4-dihydro-2,2-dimethyl-2H-1-benzopyran-3R-ol (hereinafter referred to as Compound A). It has been found that the compounds of WO 92/22293 bind to a novel receptor obtainable from rat forebrain tissue, as described in WO 96/18650 (SmithKline Beecham). The affinity of test compounds to the novel receptor site is assessed as follows.

Method

Whole forebrain tissue is obtained from rats. The tissue is first homogenised in buffer (usually 50mM Tris/HCl, pH 7.4). The homogenised tissue is washed by centrifugation and resuspension in the same buffer, then stored at −70° C. until used

To carry out the radioligand binding assay, aliquots of tissue prepared as above (usually at a concentration of 1-2 mg protein/ml) are mixed with aliquots of [3H]-Compound A dissolved in buffer. The final concentration of [3H]-Compound A in the mixture is usually 20nM. The mixture is incubated at room temperature for 1 hour. [3H]-Compound A bound to the tissue is then separated from unbound [3H]-Compound A by filtration through Whatman GF/B glass fibre filters. The filters are then washed rapidly with ice-cold buffer. The amount of radioactivity bound to the tissue trapped on the filters is measured by addition of liquid scintillation cocktail to the filters followed by counting in a liquid scintillation counter.

In order to determine the amount of specific binding of [3H]-Compound A, parallel assays are carried out as above in which [3H]-Compound A and tissue are incubated together in the presence of unlabelled Compound A (usually 3 μM). The amount of binding of [3H]-Compound A remaining in the presence of this unlabelled compound is defined as “non-specific” binding. This amount is subtracted from the total amount of [3H]-Compound A binding (i.e. that present in the absence of unlabelled compound) to obtain the amount of “specific” binding of [3H]-Compound A to the novel site.

The affinity of the binding of test compounds to the novel site can be estimated by incubating together [3H]-Compound A and tissue in the presence of a range of concentrations of the compound to be tested. The decrease in the level of specific [3H]-Compound A binding as a result of competition by increasing concentrations of the compound under test is plotted graphically, and non-linear regression analysis of the resultant curve is used to provide an estimate of compound affinity in terms of pKi value.

Results

Compounds of this invention were active in this test with pKi values greater than 6. For example, compounds of Examples 4-6, 9-12, 14-28, 45-51, 55-60 gave pKi values of greater than 7.

2. MEST Test

The maximal electroshock seizure (MEST) threshold test in rodents is particularly sensitive for detecting potential anticonvulsant properties ¹. In this model, anticonvulsant agents elevate the threshold to electrically-induced seizures whilst proconvulsants lower the seizure threshold.

Method for mouse model

Mice (naive male, Charles River, U.K. CD-1 strain, 25-30g) are randomly assigned to groups of 10-20 and dosed orally or intraperitoneally at a dose volume of 10ml/kg with various doses of compound (0.3-300 mg/kg) or vehicle. Mice are then subjected at 30 or 60 min post dose to a single electroshock (0.1 sec, 50Hz, sine wave form) administered via corneal electrodes. The mean current and standard error required to induce a tonic seizure in 50% (CC₅₀) of the mice in a particular treatment group is determined by the ‘up and down’ method of Dixon and Mood (1948)². Statistical comparisons between vehicle- and drug-treated groups are made using the method of Litchfield and Wilcoxon (1949)³.

In control animals the CC₅₀ is usually 14-18 mA. Hence the first animal in the control group is subjected to a current of 16 mA. If a tonic seizure does not ensue, the current is increased for a subsequent mouse. If a tonic convulsion does occur, then the current is decreased, and so on until all the animals in the group have been tested.

Studies are carried out using a Hugo Sachs Electronik Constant Current Shock Generator with totally variable control of shock level from 0 to 300 mA and steps of 2 mA are usually used.

Method for rat model

The threshold for maximal (tonic hindlimb extension) electroshock seizures in male rats (Sprague Dawley, 80-150 g, 6 weeks old) was determined by a Hugo Sachs Electronik stimulator which delivered a constant current (0.3 sec duration; from 1-300mA in steps of 5-20mA). The procedure is similar to that outlined above for mouse and full details are as published by Upton et al.⁴

The percentage increase or decrease in CC₅₀ for each group compared to the control is calculated.

Drugs are suspended in 1% methyl cellulose.

REFERENCES

1. Loscher, W. and Schmidt, D. (1988). Epilepsy Res., 2, 145-181

2. Dixon, W.J. and Mood. A.M. (1948). J. Amer. Stat. Assn., 43, 109-126

3. Litchfield, J.T. and Wilcoxon, F.(1949). J. Pharmacol. exp. Ther., 96, 99-113

4. N. Upton, T.P. Blackbum, C.A. Campbell, D. Cooper, M.L. Evans, H.J. Herdon, P.D. King, A.M. Ray, T.O. Stean, W.N. Chan, J.M. Evans and M. Thompson. (1997). B.J. Pharmacol., 121, 1679-1686

Results for rat MEST

Compounds of this invention dosed by the oral route as a suspension in methyl cellulose and tested one hour post dosing show an increase in seizure threshold. For example, the product of Examples 10, 11, 16, 17, and 23 showed a statistically significant increase (152, 92, 336, 403, and 107% respectively) when examined at a dose of 2 mg/kg p.o.

1. A compound of formula (Ia) or pharmaceutically acceptable salt thereof: 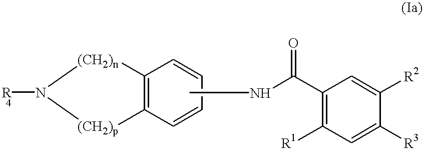
where n and p are independently integers from 1 to 4 and (n+p) is from 2 to 5;
R¹ is hydrogen, C₃₋₆cycloalkylO— or C₃₋₆cycloalkyl C₁₋₄alkylO—;
R² is hydrogen, halogen, CN, N₃, trifluoromethyldiazirinyl, C₁₋₆perfluoroalkyl, CF₃O—, CF₃S—, CF₃CO—, C₁₋₆alkyl, C₃₋₆cycloalkyl, CF₃SO₂—, C₃₋₆cycloalkyl—C₁₋₄alkyl—, C₁₋₆alkylO—, C₁₋₆alkylCO—, C₃₋₆cycloalkylCO—, C₃₋₆cycloalkyl—C₁₋₄alkylCO—, phenyl, phenoxy, benzyloxy, benzoyl, phenyl—C₁₋₄alkyl—, C₁₋₆alkylS—, C₁₋₆alkylSO₂—, (C₁₋₄alkyl)₂NSO₂, (C₁₋₄alkyl)NHSO₂, (C₁₋₄alkyl)₂NCO—,(C₁₋₄alkyl)NHCO—;or CONH₂
R³ is hydrogen, halogen, NO₂, CN, N₃, trifluoromethyldiazirinyl, C₁₋₆ alkylO—, C₁₋₆ alkylS—, C₁₋₆ alkyl, C₁₋₆ hydroxyalkyl, C₃₋₆cycloalkyl, C₃₋₆cycloalkyl—C₁₋₄alkyl—, C₁₋₆alkenyl, C₁₋₆alkynyl, CF₃CO—, C₁₋₆alkylCO—, C₃₋₆cycloalkylCO—, C₃₋₆cycloalkyl—C₁₋₄alkylCO—, phenyl, phenoxy, benzyloxy, benzoyl, phenyl—C₁₋₄alkyl—, or —NR⁵R⁶ where R⁵ is hydrogen or C₁₋₄ alkyl, and R⁶ is hydrogen, C₁₋₄alkyl, —CHO, —CO₂C₁₋₄alkyl or —COC₁₋₄alkyl; or
R² and R³ together form a carbocyclic ring that is unsaturated or saturated and unsubstituted or substituted by carbonyl or hydroxyl;
R⁴ is hydrogen, C₁₋₆ alkyl,optionally substituted by hydroxy or C₁₋₄alkoxy; C₁₋₆ alkenyl, or C₁₋₆ alkynyl, formyl, C₁₋₆alkylCO , C₁₋₆alkylSO₂, or CF₃CO—.

2. A compound according to claim 1, in which
R¹ is hydrogen or cyclopropylmethoxy;
R² is hydrogen, methoxy, bromo, chloro, iodo, acetyl, pivaloyl, iso-butyroyl, benzoyl, trifluoromethyl, trifluoroacetyl, methanesulfonyl, n-propylsulfonyl, isopropylsulfonyl, dimethylsulfamoyl, CF₃SO₂—, cyano or perfluoroethyl;
R³ is hydrogen, methyl, ethyl, n-butyl, iso-propyl, t-butyl, phenyl; methoxy, ethoxy, iso-propoxy, n-butoxy, phenoxy, benzyloxy, amino, acetylamino, nitro, benzoyl, iodobenzoyl, chloro or azido; or
R² and R³ form a benzene, cyclopentane or cyclopentanone ring;
R⁴ is hydrogen, methyl, ethyl, propyl, acetyl, trifluoromethylcarbonyl or methanesulfonyl.

3. A compound of formula (Ib) or pharmaceutically acceptable salt thereof: 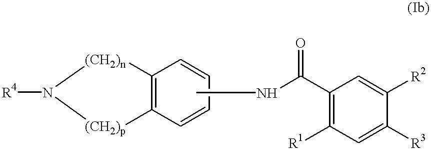
where n and p are independently integers from 1 to 4 and (n+p) is from 2 to 5;
R¹ is hydrogen, C₁₋₆alkylO—, C₃₋₆cycloalkylO— or C₃₋₆cycloalkyl C₁₋₄alkylO—;
R² is CF₃SO₂—,C₂₋₆perfluoroalkyl, (C₁₋₄alkyl)₂NCO—,(C₁₋₄alkyl)NHCO—;or CONH₂
R³ is hydrogen, halogen, NO₂, CN, N₃, trifluoromethyldiazirinyl, C₁₋₆ alkylO—, C₁₋₆ alkylS—, C₁₋₆ alkyl, C₁₋₆ hydroxyalkyl C₃₋₆cycloalkyl, C₃₋₆cycloalkyl—C₁₋₄alkyl—, C₁₋₆alkenyl, C₁₋₆alkynyl, CF₃CO—, C₁₋₆alkylCO—, C₃₋₆cycloalkylCO—, C₃₋₆cycloalkyl—C₁₋₄alkylCO—, phenyl, phenoxy, benzyloxy, benzoyl, phenyl—C. ₁₋₄alkyl—, or —NR⁵R⁶ where R⁵ is hydrogen or C₁₋₄ alkyl, and R⁶ is hydrogen, C₁₋₄alkyl, —CHO, —CO₂C₁₋₄alkyl or —COC₁₋₄alkyl;
R⁴ is hydrogen, C₁₋₆ alkyl,optionally substituted by hydroxy or C₁₋₄alkoxy; C₁₋₆ alkenyl, or C₁₋₆ alkynyl, formyl, C₁₋₆alkylCO , C₁₋₆alkylSO₂, or CF₃CO—.

4. A compound according to claim 3, in which
R¹ is hydrogen, methoxy, ethoxy, n-propoxy or cyclopropylmethoxy;
R² is CF₃SO₂—, methanesulfonyl or perfluoroethyl;
R³ is hydrogen, methyl, ethyl, n-butyl, iso-propyl, t-butyl, phenyl; methoxy, ethoxy, iso-propoxy, n-butoxy, phenoxy, benzyloxy, amino, acetylamino, nitro, benzoyl, iodobenzoyl, chloro or azido;
R⁴ is hydrogen, methyl, ethyl, propyl, acetyl, trifluoromethylcarbonyl or methanesulfonyl.

5. A compound of formula (Ic) or pharmaceutically acceptable salt thereof: 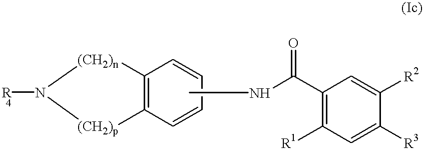
where n and p are independently integers from 1 to 4 and (n+p) is from 2 to 5;
R¹ is hydrogen, C₁₋₆alkylO—, C₃₋₆cycloalkylO— or C₃₋₆cycloalkyl C₁₋₄alkylO—;
R² and R³ together form a carbocyclic ring that is unsaturated or saturated and unsubstituted or substituted by carbonyl or hydroxyl;
R⁴ is hydrogen, C₁₋₆ alkyl,optionally substituted by hydroxy or C₁₋₄alkoxy; C₁₋₆ alkenyl, or C₁₋₆ alkynyl, formyl, C₁₋₆alkylCO, C₁₋₆alkylSO₂, or CF₃CO—.

6. A compound according to claim 5, in which
R¹ is hydrogen, methoxy, ethoxy, n-propoxy or cyclopropylmethoxy;
R² and R³ form a benzene, cyclopentane or cyclopentanone ring;
R⁴ is hydrogen, methyl, ethyl, propyl, acetyl, trifluoromethylcarbonyl or methanesulfonyl.

7. A compound of formula (Id) or pharmaceutically acceptable salt thereof: 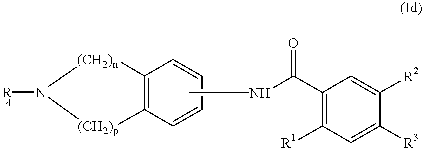
where n and p are independently integers from 1 to 4 and (n+p) is from 2 to 5;
R¹ is hydrogen, C₁₋₆alkylO—, C₃₋₆cycloalkylO— or C₃₋₆cycloalkyl C₁₋₄alkylO—;
R² is hydrogen, halogen, CN, N₃, trifluoromethyldiazirinyl, C₁₋₆perfluoroalkyl, CF₃O—, CF₃S—, CF₃CO—, CF₃SO₂—,C₁₋₆alkyl, C₃₋₆cycloalkyl, C₃₋₆cycloalkyl—C₄alkyl—, C₁₋₆alkylO—, C₁₋₆alkylCO—, C₃₋₆cycloalkylCO—, C₃₋₆cycloalkyl—C₁₋₄alkylCO—, phenyl, phenoxy, benzyloxy, benzoyl, phenyl—C₁₋₄alkyl—, C₁₋₆alkylS—, C₁₋₆alkylSO₂—, (C₁₋₄alkyl)₂NSO₂, (C₁₋₄alkyl)NHSO₂, (C₁₋₄alkyl)₂NCO—, (C₁₋₄alkyl)NHCO—;or CONH₂
R³ is hydrogen, halogen, NO₂, CN, N₃, trifluoromethyldiazirinyl, C₁₋₆ alkylO—, C₁₋₆ alkylS—, C₁₋₆ alkyl, C₁₋₆ hydroxyalkyl C₃₋₆cycloalkyl, C₃₋₆cycloalkyl—C₁₋₄alkyl—, C₁₋₆alkenyl, C₁₋₆alkynyl, CF₃CO—, C₁₋₆alkylCO—, C₃₋₆cycloalkylCO—, C₃₋₆cycloalkyl—C₁₋₄alkylCO—, phenyl, phenoxy, benzyloxy, benzoyl, phenyl—C₁₋₄alkyl—, or —NR⁵R⁶ where R⁵ is hydrogen or C₁₋₄ alkyl, and R⁶ is hydrogen, C₁₋₄alkyl, —CHO, —CO₂C₁₋₄alkyl or —COC₁₋₄alkyl; or
R² and R³ together form a carbocyclic ring that is unsaturated or saturated and unsubstituted or substituted by carbonyl or hydroxyl;
R⁴ is C₁₋₆ alkyl substituted by hydroxy or C₁₋₄alkoxy; formyl, C₁₋₆alkylCO, C₁₋₆alkylSO₂,or CF₃CO—.

8. A compound according to claim7 in which
R¹ as hydrogen, methoxy, ethoxy, n-propoxy or cyclopropylmethoxy;
R² as hydrogen, methoxy, bromo, chloro, iodo, acetyl, pivaloyl, CF₃SO₂—, iso-butyroyl, benzoyl, trifluoromethyl, trifluoroacetyl, methanesulfonyl, n-propylsulfonyl, isopropylsulfonyl, dimethylsulfamoyl, cyano or perfluoroethyl;
R³ as hydrogen, methyl, ethyl, n-butyl, iso-propyl, t-butyl, phenyl; methoxy, ethoxy, iso-propoxy, n-butoxy, phenoxy, benzyloxy, amino, acetylamino, nitro, benzoyl, iodobenzoyl, chloro or azido; or
R² and R³ form a benzene, cyclopentane or cyclopentanone ring;
R⁴ as acetyl, trifluoroacetyl or methanesulfonyl.

9. A compound according to any one of the preceding claims which is a compound described in any one of the foregoing Examples.

10. A compound of formula (I) as hereinbefore defined, substantially as hereinbefore described in any one of the Examples.

11. A pharmaceutical composition for use in the treatment and/or prophylaxis of anxiety, mania, depression, panic disorders and/or aggression, disorders associated with a subarachnoid haemorrhage or neural shock, the effects associated with withdrawal from substances of abuse such as cocaine, nicotine, alcohol and benzodiazepines, disorders treatable and/or preventable with anti-convulsive agents, such as epilepsy including post-traumatic epilepsy, Parkinson's disease, psychosis, migraine, cerebral ischaemia, Alzheimer's disease and other degenerative diseases such as Huntingdon's chorea, schizophrenia, obsessive compulsive disorders (OCD), neurological deficits associated with AIDS, sleep disorders (including circadian rhythm disorders, insomnia & narcolepsy), tics (e.g. Giles de la Tourette's syndrome), traumatic brain injury, tinnitus, neuralgia, especially trigeminal neuralgia, neuropathic pain, dental pain, cancer pain, inappropriate neuronal activity resulting in neurodysthesias in diseases such as diabetes, multiple sclerosis (MS) and motor neurone disease, ataxias, muscular rigidity (spasticity), temporomandibular joint dysfunction and/or amyotrophic lateral sclerosis (ALS). which comprises a compound as claimed in any one of claims 1 to 10, or a pharmaceutically acceptable salt or solvate thereof, and a pharmaceutically acceptable carrier.

12. A method of treatment and/or prophylaxis of anxiety, mania, depression, panic disorders and/or aggression, disorders associated with a subarachnoid haemorrhage or neural shock, the effects associated with withdrawal from substances of abuse such as cocaine, nicotine, alcohol and benzodiazepines, disorders treatable and/or preventable with anti-convulsive agents, such as epilepsy including post-traumatic epilepsy, Parkinson's disease, psychosis, migraine, cerebral ischaemia, Alzheimer's disease and other degenerative diseases such as Huntingdon's chorea, schizophrenia, obsessive compulsive disorders (OCD), neurological deficits associated with AIDS, sleep disorders (including circadian rhythm disorders, insomnia & narcolepsy), tics (e.g. Giles de la Tourette's syndrome), traumatic brain injury, tinnitus, neuralgia, especially trigeminal neuralgia, neuropathic pain, dental pain, cancer pain, inappropriate neuronal activity resulting in neurodysthesias in diseases such as diabetes, multiple sclerosis (MS) and motor neurone disease, ataxias, muscular rigidity (spasticity), temporomandibular joint dysfunction and/or amyotrophic lateral sclerosis (ALS), comprising administering to the sufferer in need thereof an effective or prophylactic amount of a compound as claimed in any one of claims 1 to 10, or a pharmaceutically acceptable salt or solvate thereof.

13. Use of a compound as claimed in any one of claims 1 to 10, or a pharmaceutically acceptable salt or solvate thereof, for the manufacture of a medicament for the treatment and/or prophylaxis of anxiety, mania, depression, panic disorders and/or aggression, disorders associated with a subarachnoid haemorrhage or neural shock, the effects associated with withdrawal from substances of abuse such as cocaine, nicotine, alcohol and benzodiazepines, disorders treatable and/or preventable with anti-convulsive agents, such as epilepsy including post-traumatic epilepsy, Parkinson's disease, psychosis, migraine, cerebral ischaemia, Alzheimer's disease and other degenerative diseases such as Huntingdon's chorea, schizophrenia, obsessive compulsive disorders (OCD), neurological deficits associated with AIDS, sleep disorders (including circadian rhythm disorders, insomnia & narcolepsy), tics (e.g. Giles de la Tourette's syndrome), traumatic brain injury, tinnitus, neuralgia, especially trigeminal neuralgia, neuropathic pain, dental pain, cancer pain, inappropriate neuronal activity resulting in neurodysthesias in diseases such as diabetes, multiple sclerosis (MS) and motor neurone disease, ataxias, muscular rigidity (spasticity), temporomandibular joint dysfunction and/or amyotrophic lateral sclerosis (ALS).
